(19) United States
(12) Patent Application Publication
(10) Pub. No.: US 2004/0259790 A1
(43) Pub. Date: Dec. 23, 2004

(54) Methods for identifying and administering agents that bias the immune response via dendritic cells

(76) Inventors: Bali Pulendran, Alpharetta, GA (US); Sudhanshu Agrawal, Irvine, CA (US); Stephanie Maree Dillon, Atlanta, GA (US)

Correspondence Address: MANDEL & ADRIANO, 55 SOUTH LAKE AVENUE, SUITE 710, PASADENA, CA, 91101, US

(21) Appl. No.: 10/769,635

(22) Filed: Jan. 30, 2004

Related U.S. Application Data
(60) Provisional application No. 60/443,692, filed on Jan. 30, 2003.
(60) Provisional application No. 60/516,169, filed on Oct. 31, 2003.

(51) Int. Cl. A61K 38/17

(52) U.S. Cl. 514/12; 424/93.71

(57) ABSTRACT

The invention provides a method of regulating a Th2 immune response which comprises contacting a cell with an amount of a molecule effective to modulate an ERK ½ pathway and/or a c-FOS pathway in the cell so as a to regulate the TH2 immune response, which molecule is any of (a) an agonist of a TLR2 or a TLR2 variant; (b) an agonist of an intracellular pathway that is initiated by activation of a TLR2; (c) an agonist of an intracellular pathway that is initiated by activation of a receptor activated by SEA; (d) an antagonist of an intracellular pathway that opposes TLR2 signaling or activation; (e) an agonist of an ERK ½ pathway; (f) an antagonist of a p38 pathway; (g) an antagonist of a JNK ½ pathway; or (h) an agonist of the c-FOS pathway, or a molecule that induces c-Fos gene expression, c-Fos messenger RNA stability, c-Fos protein induction, c-Fos protein stability, or c-Fos protein phosphorylation.

This application claims the benefit of the filing dates of U.S. Ser. No. 60/443,692, filed Jan. 30, 2003 and U.S. Ser. No. 60/516,169, filed Oct. 31, 2003, the contents of which are incorporated by reference into the present application in their entireties.

Throughout this application, various publications are referenced within parentheses. The disclosures of these publications are hereby incorporated by reference herein in their entireties.

The work described here was supported, at least in part, by grants from the National Institutes of Health (grant numbers AI48638-01 and DK57665-01). The United States government may, therefore, have certain rights in the invention. Throughout this application various publications are referenced. The disclosures of these publications in their entireties are hereby incorporated by reference into this application in order to more fully describe the state of the art to which this invention pertains.

### FIELD OF INVENTION

This invention relates to the field of immunology, and more particularly to methods for biasing the immune response towards different T helper cell (e.g, Th) responses in individuals who have an immune-related disease or condition.

### BACKGROUND OF THE INVENTION

The immune system is a remarkably adaptive and versatile system that can generate distinct (e.g., allergens, pathogens, and other non-self molecules), but the molecular signals that direct the immune system along one course or another are largely unknown. This has hampered efforts to develop therapeutic agents that can modulate the immune response and thereby treat patients with allergies, autoimmune disease, and other immune-related conditions (e.g., cancer).

The adaptive immune system has evolved different types of immune responses against distinct pathogens. For example, immune responses against T-cell-dependent antigens display staggering heterogeneity with respect to the cytokines made by T-helper cells and the class of antibody secreted by B cells (Mosmann, T. R. & Coffinan, R. L., Annu. Rev. Immunol. 7:145-173 (1989); Seder, R. A. & Paul, W. E., Annu. Rev. Immunol 12:635-673 (1994); Szabo S J, Sullivan B M, Peng S L, Glimcher L H., Annu Rev Immunol. 21:713-58 (2003); Murphy, K. M. et al., Annu. Rev. Immunol., 18:451-494 (2000)). Thus, in response to viruses or intracellular microbes, CD4+ T-helper (Th) cells differentiate into Th1 cells, which produce IFNγ, and activate macrophages to produce mediators such as NO and TNF, which kill the intracellular pathogen.

In contrast, helminths parasites induce the differentiation of Th2 cells, whose cytokines (principally IL-4, IL-5, IL-10 and IL-13) induce IgE and eosinophil-mediated destruction of the pathogens (Mosmann, T. R. & Coffinan, R. L., Annu. Rev. Immunol. 7:145-173 (1989); Seder, R. A. & Paul, W. E., Annu. Rev. Immunol 12:635-673 (1994); Szabo S J, Sullivan B M, Peng S L, Glimcher L H., Annu Rev Immunol. 21:713-58 (2003); Murphy, K. M. et al., Annu. Rev. Immunol., 18:451-494 (2000)). At the individual T-cell level, considerable heterogeneity of cytokine profiles can be seen with T-cell clones, raising the possibility that the canonical Th1 and Th2 global phenotypes only represent two polar extremes of all possible single cell phenotypes (Kelso A., Immunol. Today, 16:374-379 (1995)).

While there is much knowledge about the cytokines produced early in the response, and the transcription factors that determine Th polarization in T cells (Szabo S J, Sullivan B M, Peng S L, Glimcher L H., Annu Rev Immunol. 21:713-58 (2003); Murphy, K. M. et al., Annu. Rev. Immunol., 18:451-494 (2000)), the early “decision-making mechanisms,” which result in a given type of immune response are poorly understood.

Emerging evidence suggests that the type of response is a complex function of several determinants, including the dendritic cell (DC) subset, the nature of the microbial stimuli, the local microenvironment and cytokines (Kalinski, P., C. M. Hilkens, E. A. Wierenga, M. L. Kapsenberg, 2000, Immunol Today, 12:561; Pulendran, B., K. Palucka, and J. Banchereau, 2001, Science, 293:253; Shortman, K. and Y. J. Liu, 2002, Nature Reviews Immunol, 2:151).

Several recent observations bear on this problem. Distinct types of dendritic cell (DC) subsets can differentially induce Th1 and Th2 responses (Maldonado-Lopez, R., T. et al., J. Exp. Med., 189:587-592 (1999); Pulendran, B. et al., Proc. Natl. Acad. Sci. USA, 96:1036-1041 (1999); Rissoan, M. C. et al., Science, 283:1183-1186 (1999)). For example, in mice, CD8α+ DCs can elicit Th1 cells, while CD8α− DCs can induce Th2/Th0 cells (Maldonado-Lopez, R., T. et al., J. Exp. Med., 189:587-592 (1999); Pulendran, B. et al., Proc. Natl. Acad. Sci. USA, 96:1036-1041 (1999)).

The dose of antigen can play an important role in influencing the Th1/Th2 balance (Boonstra, A. C. et al., J. Exp. Med., 197:101-109 (2003)).

Different microbial stimuli signal through distinct pattern recognition receptors on antigen presenting cells (APC) (Medzhitov, R., & Janeway, C., Immuno.l Rev., 173:89-97 (2000); Aderem, A., & Ulevitch, R. J. (2000). Nature, 406:782-787; Akira, S., Takeda, K., & Kaisho, T., Nat. Immunol., 2:675-680 (2001)). For example, LPS from E. coli signals through the Toll-4 receptor (TLR-4) (Poltorak, A. et al., Science, 282:2085-2088 (1998)), while TLR-2 appears to have a broad spectrum of ligands, including peptidoglycan of Staphylococcus aureus (Takeuchi, O. et al., Immunity, 11:443-451 (1999)), lipoproteins from M tubercolosis (Brightbill, H. D. et al., Science, 285:732-736 (1999); Aliprantis, A. O. et al., Science, 285:736-739 (1999)), and Sacharomyces cerevisiae zymosan (Underhill, D. M. et al., Nature, 401:811-815 (1999)).

Different microbial stimuli differentially activate DCs to elicit distinct classes of immune responses (Kalinski, P., Hilkens, C. M., Wierenga, E. A. & Kapsenberg, M. L., Immunol. Today., 12:561-567 (2000); Moser, M. & Murphy, K. M., Nat. Immunol., 3:199-205 (2000); Pulendran, B., Palucka, K. & Banchereau, J., Science, 293:253-256 (2001a); Liu, Y. J., Cell, 106:259-262 (2001); Shortman, K. & Liu, Y. J., 2002, Nat. Rev. Immunol., 2:151-161; Rescigno, M., Trends Microbiol., 10:425-461 (2002)). For example, toxoplasma extracts (Reis e Sousa C, Hieny S, Scharton-Kersten T, Jankovic D, Charest H, Germain R N, Sher A., J Exp Med, 186:1819-29 (1997)) and E. coli LPS stimulates IL-12(p70) production in CD8α+ DCs and primes a Th1 response (Pulendran, B. et al., J. Immunol., 167:5067-5076 (2001b)), and certain viruses induce IFN-α from plasmacytoid DCs and stimulate Th1 responses (Kadowaki, N., Antonenko, S., Lau, J. Y., & Liu, Y. J., J. Exp. Med., 192:219-226 (2000); Cella, M. D. et al., Nat. Med., 5:919-923 (1999)).

In contrast, schistosome egg antigens (SEA) (MacDonald, A. S., Straw, A. D., Bauman, B., & Pearce, E. J., J. Immunol., 167:1982-1988 (2001)), filarial antigens (Whelan, M., et al., J. Immunol., 15:6453-6460 (2000)), cholera toxin (Braun, M. C., He, J., Wu, C. J., & Kelsall, B. L., J. Exp. Med., 189:541-552 (1999)), lipoxins stimulated by toxoplasma (Aliberti J, Hieny S, Reis e Sousa C, Serhan C N, Sher A., (2002). Nat. Immunol., 3:76-82), certain forms of Candida albicans (d'Ostiani, C. F. et al., J. Exp. Med., 191:1661-1674 (2000)), or highly purified preparations of P. gingivalis LPS (Pulendran, B. et al., J. Immunol., 167:5067-5076 (2001b)), do not stimulate IL-12(p70), and favor Th2-like responses. Interestingly, a recent report suggests that P. gingivalis LPS signals via TLR 2 in murine macrophages (Hirschfeld, M. et al., Infect. Immun., 69:1477-1482 (2001)). CpG DNA induces IL-12(p70) in DCs and elicits Th1 responses (Krieg AM, 2002, Ann. Rev. Immunol., 20:709). P. gingivalis LPS (Pulendran B., et al., 2001, J. Immunol., 167:5067), fail to induce IL-12(p70), and stimulate Th2-like responses.

Thus, although different DC subsets may have some intrinsic potential to preferentially induce Th1 or Th2 responses DCs also display considerable functional plasticity in response to signals from microbes and the local microenvironments. The nature of the pathogen-recognition receptors, which enable DCs to sense such diverse stimuli are only beginning to be understood.

Recent efforts have focused on the Toll-like receptors (TLRs), which have broad specificity for conserved molecular patterns shared by large groups of pathogens (Medzhitov, R., and C. Janeway, Jr., 2000, Immunol Rev., 173:89; Aderem, A., and R. J. Ulevitch, 2000, Nature, 406:782; Akira, S., K. Takeda, T. Kaisho, 2001, Nature Immunol., 2:675). The expression of different TLRs on DCs, enable them to discriminate between different stimuli. For example, E. coli LPS signals through TLR4, zymosan, and peptidoglycans from Staphylococcal aureus signal through TLR2, CpG rich bacterial DNA signal through TLR9, and bacterial flagellin signal through TLR5 (Medzhitov, R., and C. Janeway, Jr., 2000, Immunol Rev., 173:89; Aderem, A., and R. J. Ulevitch, 2000, Nature, 406:782; Akira, S., K. Takeda, T. Kaisho, 2001, Nature Immunol., 2:675). It has been suggested that signaling through any of the TLRs, instruct DCs to preferentially stimulate Th1 responses (Medzhitov, R., and C. Janeway, Jr., 2000, Immunol Rev., 173:89). Although, P. gingivalis LPS, a putative TLR2 agonist (Hirschfeld M., J. J. Weis, V. Toshchakov, C. A. Salkowski, M. J. Cody, N. Ward, D. C. Qureshi, S. M. Michalek, S. N. Vogel, 2001, Infect. Immun., 69:1477) favors Th2 responses (Pulendran B., et al., 2001, J. Immunol., 167:5067), it is not clear whether this is a characteristic of all TLR2 agonists, or if this is simply a peculiarity of P. gingivalis LPS. If indeed, signaling via different TLRs instruct DCs to elicit distinct Th responses, then the intracellular signaling pathways, which mediate such different outcomes, are not known. Here we demonstrate that signaling via distinct TLRs conditions human monocyte-derived DCs to bias towards different Th responses, via differential modulation of distinct components of the MAP-kinase signaling pathway.

In this context, it is unclear whether signaling through distinct TLRs can shift the balance towards the Th2 phenotype. Although, our recent work with P. gingivalis LPS suggests that signaling through TLR-2 can stimulate Th2-like responses (Pulendran, B. et al., J. Immunol., 167:5067-5076 (2001b)), it is not clear whether this is characteristic of all TLR-2 ligands, or whether this is a unique feature of P. gingivalis LPS. Here we address these issues, using a synthetic TLR 2 ligand, Pam₃cys-Ser-(Lys)₄ ({S-[2,3-bis(palmitoyloxy)-(2-RS)-propyl]-N-paInitoyl-R-Cys-S-Ser-Lys4-OH)} or Pam-3-cys] (Takeuchi, et al., 2001 International Immunology 13:933-940; Akira, S., Takeda, K., & Kaisho, T., Nat. Immunol., 2:675-680 (2001)), and highly purified preparations of E. coli LPS. Our data suggest that signaling via distinct TLR 2 conditions DCs to modulate the Th balance towards the Th2 pathway, via a mechanism involving enhanced induction of ERK and the early growth transcription factor, c-Fos.

### SUMMARY OF THE INVENTION

The present invention is based, in part, on our discovery that dendritic cells (e.g., DCs—the bone marrow-derived leukocytes that take up and present antigens to T cells), toll-like receptors (TLRs), and components of the intracellular signaling pathways triggered by TLRs are all targets that, when contacted with agents that either stimulate or inhibit as described herein, modulate the response of T-helper (Th) cells. More specifically, our work demonstrates that signaling via distinct TLRs conditions DCs to elicit different Th responses via preferential activation of distinct components of the MAP-kinase signaling pathway. Thus, the systems and agents described herein can be used to identify pharmaceutical agents that can be used to effect or produce adaptive immunity in the immune therapy of e.g., allergy, autoimmunity, transplantation, and cancer.

Generally, the methods of the invention that concern treating a patient can be carried out by administering to the patient an agent or cell (the agents and cells are described further below) that biases the immune response toward a particular Th response (e.g., a Th1 or Th2 response (these responses are widely believed to constitute the extremes of the Th response), a Th0, or a T-regulatory response (these responses are considered more neutral; as these responses are toward the middle of the response spectrum, they can benefit patients who have, or who may develop, immune-related diseases associated with responses either the Th1 or Th2 end of the spectrum)). Th1 and Th2 are sometimes abbreviated as T_{H}1 and T_{H}2, respectively; in any event, they are terminally differentiated subclasses of T-helper cells that secrete a restricted repertoire of cytokines.

As these types of responses are associated with various immune-related diseases, modulating the response is an effective way to ameliorate the disease process. For example, certain autoimmune diseases (e.g., diabetes, rheumatoid arthritis, multiple sclerosis, psoriasis, and systemic lupus erythrematosis) are associated with an exuberant Th1 response. Thus, strategies that bias the immune response away from the Th1 response and toward the less harmful Th2 response (or that decrease or inhibit the Th1 response) benefit patients who have one of these diseases or who are at risk for developing them. Similarly, other conditions (e.g., allergy, asthma, and chronic obstructive pulmonary disease (COPD (e.g., emphysema or chronic bronchitis)) are associated with exuberant Th2 responses. Thus, strategies that bias the immune response away from the Th2 response and toward the less harmful Th1 response (or that decrease or inhibit the Th2 response) benefit patients who have one of these diseases or who are at risk for developing them. As the phenotypes of T-helper cells are defined well enough to be categorized and placed on the response spectrum, any patient's response status can be determined and monitored over time (e.g., over the course of a disease or following the initiation of a treatment regime (whether that regime is specifically aimed at biasing the immune response, (e.g., as described herein), treating the disease in some other way(s), or both)). Another class of patients that can be treated according to the methods of the invention are those suffering from sepsis.

The methods that involve patient treatment can be carried out by administering an agent to a patient directly (i.e., the agent, regardless of its mechanism of action, can be appropriately formulated as a pharmaceutical composition and administered to the patient). Alternatively, or in addition, one or more agents can be delivered indirectly (i.e., the patient can receive cells or cell-based compositions in which the cells were treated in culture with an agent that biases the immune response). The agents include: (1) agonists and antagonists of TLRs (e.g., TLR-2, TLR-3, TLR-4, TLR-5, TLR-7, and TLR-9), (2) agonists and antagonists of the receptor(s) activated by schistosome egg antigen (SEA), (3) molecules that stimulate or inhibit the expression or activity of a component of an intracellular signaling pathway that transduces the signal generated by activation of either of these types of receptors, and (4) agents that stimulate or inhibit a transcription factor that is induced or stabilized by one or more of these signaling pathways. One or more of these types of agents can be administered alone, in combination with one another, or in combination with other therapies for autoimmune disease or cancer.

The methods can be used to treat a patient (a human patient or other animal that experiences immune-related disorders) who would benefit from an immune response biased toward the generation of T-helper cells of subclass 2 (Th2 cells), Th0 cells, or T-regulatory cells (i.e., a patient who has, or who may develop, a disease or condition caused by (or otherwise adversely associated with) a Th1 cell response). One can carry out these methods by administering to the patient or contacting a cell expressing a TLR with: (a) an agonist of TLR-2 or a receptor activated by SEA; or (b) an agent that stimulates an intracellular signaling pathway initiated by activation of TLR-2 or the receptor activated by SEA.

The agonist can be an exogenous or endogenous ligand, many of which are known in the art. The novel screening methods described below, particularly those that feature detecting TLR binding or activation, can be used to identify other ligands (whether naturally occurring molecules, fragments or derivatives thereof, antibodies, other peptides or protein-containing complexes, or synthetic ligands). For example, exogenous ligands of TLR-2 include LPS (lipopolysaccharide; a component of the outer membrane of Gram-negative bacteria), yeast-particle zymosan, bacterial peptidoglycans, lipoproteins from bacteria and mycoplasmas, and GPI anchor from Trypanosoma cruzi; endogenous ligands include heat shock (or “stress”) proteins (e.g., an Hsp60 from, for example, a bacterial or mycobacterial pathogen) and surfactant protein-A. Exogenous ligands of TLR-3 include poly(I:C) (viral dsNRA); exogenous ligands of TLR-4 include LPS, and respiratory syncytial virus (endogenous ligands include stress proteins such as an Hsp60 or Hsp70, saturated fatty acids, unsaturated fatty acids, hyaluronic acid and fragments thereof, and surfactant protein-A). Flagellin is an exogenous ligand of TLR-5. CpG (cytosine-guanine repeat) DNA and dsDNA are exogenous and endogenous ligands, respectively, of TLR-9. See Zuany-Amorim et al., Nature Reviews 1:797-807, 2002, and Takeda et al., Ann. Rev. Immunol. 21:355-376, 2003.

As noted above, patients can also be treated with cells or cell-based therapies. For example, to bias the immune response toward the generation of Th2 cells, Th0 cells, or T-regulatory cells (i.e., away from a Th1 response), the patient can receive dendritic cells (or antigen-presenting cells) treated in culture with (i) an agonist of a TLR-2, (ii) an agonist of the receptor activated by SEA, (iii) an agent that stimulates an intracellular signaling pathway initiated by activation of TLR-2, or (iv) an agent that stimulates an intracellular signaling pathway initiated by activation of a receptor activated by SEA. Alternative, or in addition, the patient can receive T cells (e.g., syngeneic T cells) stimulated in culture with dendritic cells treated as described immediately above. The amount of the agonist or the agent administered to the patient, or the number of the dendritic cells or the T cells administered to the patient, should be sufficient to expand the population of Th2 cells, Th0 cells, or T-regulatory cells in the patient. Methods of culturing antigen-presenting cells and T cells are known in the art (see also, the Examples below).

Any of the methods in which the immune response is biased toward Th2 can be reinforced by carrying them out together with a method that biases the response away from Th1. Thus, any of the methods described above can be carried out in concert with any of the methods that inhibit the generation of Th1 cells (e.g. methods in which any, or any combination of, TLR-3, TLR-4, TLR-5, TLR-7, or TLR-9 are antagonized; methods in which a signaling pathway downstream from these receptors is inhibited; or methods in which cells (e.g., dendritic cells or T cells) treated in culture with such receptor or pathway antagonists are administered to the patient). The converse is also true. Any of the methods in which the immune response is biased toward Th1 can be reinforced by carrying them out in concert with any of the methods that bias the response away from Th2 (e.g., methods in which Th2 antagonists are administered or in which the pathways that mediate TLR-2 receptor signaling are inhibited).

In other embodiments, the invention features methods of treating a patient who would benefit from an immune response biased toward the generation of T-helper cells of subclass 1 (Th1 cells). One can carry out these methods by administering to the patient: (a) an agonist of a Toll-like receptor of type 3, 4, 5, 7, or 9 (TLR-3, TLR-4, TLR-5, TLR-7, or TLR-9, respectively); (b) an agent that stimulates an intracellular signaling pathway initiated by agonists of TLR-3, TLR-4, TLR-5, TLR-7, or TLR-9; (c) dendritic cells treated in culture with an agonist of TLR-3, TLR-4, TLR-5, TLR-7, or TLR-9 or an agent that stimulates an intracellular signaling pathway initiated by activation of one of these receptors; and/or (d) syngeneic T cells stimulated in culture with dendritic cells treated as described in (c). The amount of the agonist or the agent administered to the patient, or the number of the dendritic cells or the T cells administered to the patient should be sufficient to expand the Th1 cell population in the patient.

Other methods of treating patients who would benefit from an immune response biased toward the generation of Th1 cells can be carried out by administering to the patient (a) an agent that inhibits the expression or activity of an AP-1 transcription factor in a dendritic cell, (b) a dendritic cell treated in culture with an agent that inhibits the expression or activity of an AP-1 transcription factor, or (c) syngeneic T cells stimulated in culture with dendritic cells treated as described in (b). Here again, the amount of the agent administered to the patient, or the number of the dendritic cells or the T cells administered to the patient, should be sufficient to bias the immune response toward Th1 cells. The transcription factor can include c-fos, fos-B, or c-jun, and the agent that inhibits expression (of the transcription factor or of any component of the pathways described herein (these components are known in the art)) can be an antisense oligonucleotide or an RNAi molecule that specifically inhibits c-fos, fos-B, or c-jun expression (or the expression of a kinase, phosphatase, or other component of the signaling pathways). The inhibitory agents or antagonists discussed in the context of the present methods can also be antibodies (or variants thereof (e.g., single-chain antibodies or humanized antibodies); preferably the antibodies are monoclonal antibodies).

Also within the scope of the invention are pharmaceutically acceptable compositions that include an agonist or antagonist of TLR-2 and a carrier, excipient, or diluent; an agonist or antagonist of TLR-3, TLR-4, TLR-5, TLR-7, or TLR-9; or an agent that stimulates or inhibits a component of the signaling pathways that transduce the signal generated by receptor binding.

Another aspect of the invention encompasses screening assays. For example, the invention features a method of determining whether an agent (a broad term meant to include biological and synthetic molecules or fragments or derivatives thereof) biases the immune response toward, or away from, the generation of Th2 cells. These methods can be carried out by: (a) providing a cell that expresses a TLR-2 or a receptor activated by SEA; (b) exposing the cell to the test agent under conditions and for a time sufficient to allow the test agent to contact the cell or bind TLR-2 or bind the receptor activated by SEA; and (c) detecting receptor binding or activation (binding or activation indicating that the test agent is an agent that biases the immune response toward or away from the generation of Th2 cells). To detect reactivity or receptor binding, one can use methods known in the art to detect complex formation between the test agent and the receptor or to detect the stimulation or inhibition of an intracellular signaling pathway initiated by activation of TLR-2 or the receptor activated by SEA. The cells used in the assay can be (but are not necessarily) dendritic cells, which can be cultured under the conditions described in the present Examples. Analogous methods can be carried out to determine whether an agent biases the immune response toward, or away from, the generation of Th1 cells (here, one would provide a cell that expresses a TLR-3, TLR-4, TLR-5, TLR-7, or TLR-9).

In addition to the pharmaceutical formulations described above, the present invention features kits that include a cell that expresses a TLR and instructions for using the cell to identify TLR agonists or antagonists that, upon administration to a patient, bias the immune response toward the production of Th1 cells or Th2 cells. One or more other compositions described herein can also be combined and packaged as a useful kit.

Our studies establish, for the first time, that activating innate immune cells via TLRs does not always result in polarized Th1 responses (as previously suggested; see, e.g., Brightbill et al., Science 285:732-736, 1999; Sieling et al., J. Immunol. 170:194-200, 2003). To the contrary, TLR activation can also induce Th2 and/or Th0 responses or other more neutral immune responses. Our data also reveal a mechanism involving differentially triggered MAP-kinases, which mediate the distinct DC response to TLR ligands (at least in part). In addition, our data highlight fundamental differences in the phosphorylation and stabilization of c-Fos, which is phosphorylated and stabilized by prolonged ERK ½ signaling (Murphy et al., Nature Cell Biol. 4:556-564, 2002), and they suggest that inhibition of c-Fos results in enhanced IL-12 in response to LPS and flagellin (FIG. 5B). The failure to consistently enhance IL-12 in response to Pam3cys or SEA, suggests that suppression of IL-12 by these stimuli is very potent, and negatively regulated by additional pathways. We hypothesize that IL-12 is regulated by other members of the AP-1 family, which consists of at least 18 dimeric combinations of proteins from the Jun (c-Jun, JunB and JunD) and Fos (c-Fos, Fos-B, Fra-1 and Fra-2) families (including Jun-Jun homodimers, and Jun-Fos heterodimers). IL-12 production may also require enhanced activity of p38 and JNK1/2 and reduced activity of ERK1/2 and c-Fos. Since stimulation of DCs with E. coli LPS or flagellin satisfies all of these criteria, IL-12 would be efficiently induced with these agonists. In contrast, stimulation with Pam3cys or SEA, fails to induce strong or sustained phosphorylation of JNK1/2 and p38.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein. All publications, patent applications, patents, and other references mentioned herein are incorporated herein by reference in their entirety.

The details of one or more embodiments of the invention are set forth in the accompanying drawings and the description below. Other features, objects, and advantages of the invention will be apparent from the description and drawings, and from the claims.

### BRIEF DESCRIPTION OF THE DRAWINGS

FIGS. 1A and 1B are FACS profiles and bar graphs supporting our conclusion that different TLR ligands induce distinct DC responses. Immature, monocyte-derived DCs were cultured for 48 hours with E. coli LPS (Ec. LPS), Pam3cys, flagellin and SEA (all of which are microbial agents and some of which are known TLR ligands). FIG. 1A shows the DC response as measured by flow cytometric analyses of the expression of CD80 and CD86 (costimulatory molecules) and the maturation marker CD83. FIG. 1B shows the DC response as measured by ELISA of secreted cytokines (IL12p70, IL-10, 1L-6, and TNFα). Each dot on the histograms represents a single donor, and the Y-axis represents pg of cytokine/ml supernatant.

FIGS. 2A-2C are graphs. DCs activated with different TLR ligands stimulate distinct T-helper responses. Immature, monocyte-derived DCs were cultured, for 48 hrs, with different microbial stimuli, including various TLR ligands. Then, the DCs were washed and then cultured at graded doses, with 10⁵ naïve, allogeneic CD4+ T cells. (A) After 5 days, the T-cell proliferation was assessed by overnight [³H] thymidine labeling. Gray, black and speckled histograms represent 0, 10⁴, and 2×10³ DCs, respectively. (B) The secretion of Th1 and Th2 cytokines in culture, was assessed by ELISA. (C) The ratios of Th1/Th2 cytokines was evaluated for each of the stimuli.

FIGS. 3A and 3B. Different TLR ligands stimulate distinct magnitude and duration of MAP-kinase signaling in DCs. Immature, monocyte-derived DCs were cultured with different microbial stimuli, including various TLR ligands, for 0 minutes, 15 minutes, 1 hour and 4 hours. (A) At each time point, the expression of phosphorylated and total p38 and ERK was evaluated by ELISA. Data is presented as the fold increases in the phosphorylated to total protein ratios, relative to the 0 minute value. (B) Expression of JNK1, JNK2 and total JNK was determined by Western Blot analysis.

FIG. 4. Induction of IL-12(p70) is enhanced by p38 and JNK1/2 signaling, and suppressed by ERK1/2 signaling. Immature, monocyte-derived DCs were cultured, for 48 hours, with different microbial stimuli, including various TLR ligands, either in the presence of absence of synthetic inhibitors of p38, JNK1/2 and ERK1/2 signaling.

FIGS. 5A and 5B. Distinct TLR ligands differentially induce c-Fos, which regulates the production of IL-12(p70). (A) Flow cytometric analyses of the expression of total c-Fos and phosphorylated c-Fos, in DCs stimulated with different stimuli. (B) Effect of blocking c-Fos activity, on IL-12(p70) production.

FIG. 6. A model for “DCI/DC2” regulation by MAP-kinases and c-Fos. TLR4 or TLR5 ligands induce strong activation of p38 and JNK, but only a transient activation of ERK1/2. This results in the production of IL-12(p70), which stimulates Th1 responses. In contrast, TLR2 ligands and SEA induce sustained phosphorylation of ERK, which stabilizes c-Fos, which suppresses the production of IL-12(p70).

FIG. 7: Different TLR ligands induce distinct DC responses. Immature, monocyte-derived DCs were cultured, for 48 hrs, with different microbial stimuli. DC responses were measured as follows. (a) Flow cytometric analyses of the expression of the costimulatory molecules CD80 and CD86, and the maturation marker CD83. Blue, isotype; red, marker (b) Secretion of cytokines in the culture supernatants, measured was by ELISA. Each dot on the histograms represents a single donor. Representative of 7 experiments.

FIG. 8: DCs activated with different TLR ligands stimulate distinct T-helper responses.

Immature, monocyte-derived DCs were cultured, for 48 hrs, with different microbial stimuli, including various TLR ligands. Then, the DCs were washed and then cultured at graded doses, with 10⁵ naïve, allogeneic CD4+ T cells. (a) After 5 days, the T-cell proliferation was assessed by overnight [³H] thymidine labeling. Black, hatched and white histograms represent 0, 10⁴, and 2×10³ DCs, respectively. (b) The secretion of Th1 and Th2 cytokines in culture, was assessed by ELISA. (c) The ratios of Th1/Th2 cytokines were evaluated for each of the stimuli. Representative of 7 experiments.

FIG. 9: Distinct TLR ligands differentially stimulate ERK and JNK signaling, which regulate IL-12(p70) and IL-10. Day 6 DCs were cultured with different microbial stimuli, for 0 minutes [white bar], 0.25 hr [black bar], 1 hr [grey bar] and 4 hrs [speckled bar]. (a) At each time point, the expression of phosphorylated and total ERK was evaluated by ELISA. Data is presented as the fold increases in the phosphorylated to total protein ratios, relative to the 0 minute value. (b) Flow cytometric analyses of phosphor-ERK expression in DCs. Dotted histogram represents the staining in unstimulated DCs, and the bold histograms represent staining after stimulation. (c) Expression of JNK1, JNK2 and total JNK was determined by western blot analysis. (d) The effect of blocking p38 or JNK1/2 on IL-12p70 secretion. IL-12(p70) levels, after blocking with inhibitors, are expressed as a percentage of levels without inhibitor, (which is 100%). Representative of 5 experiments.

FIG. 10: Distinct TLR ligands differentially induce c-Fos, which regulates the production of IL-12(p70). (a) Flow cytometric analyses of the expression of total c-Fos and phosphorylated c-Fos, in DCs stimulated with different stimuli. (NO COLOR, BLACK AND WHITE FIGURES) Blue histograms indicate expression levels on unstimulated DCs, and the red histograms represent expression after stimulation. (b) Inhibition of c-fos by si RNA does not affect IL-6 induction in DCs, in response to Pam-3-cys and SEA. (c) However, this si-RNA results in a striking increase in the secretion of IL-12(p70), in response to Pam-3-cys and SEA. “Stimuli,” is DC+stimulus; “DC+siRNA” is DCs cultured with siRNA, without any stimulus; “si RNA 1, 4 and b-actin,” are DCs cultured with the respective siRNAs, and then stimulated; “mock,” represents mock transfected DCs.

FIG. 11. E. coli LPS and Pam-3-cys activate splenic CD11c⁺ CD11b⁺ and CD11c⁺ CD11b⁻ DC subsets in vivo. E. coli LPS (25 μg), Pam-3-cys (50 μg) or PBS were injected i.p. into wild type (3 per group) or TLR2−/− mice (3 per group) and the expression of costimulatory molecules determined 6 hours later. These doses induced equivalent upregulation of CD86 and I-A^{b} on both DC subsets. E. coli LPS and Pam-3-cys upregulated CD86 and I-Ab expression on both CD11c⁺ CD11b⁺ and CD11c⁺ CD11b⁻ DC subsets from wild type mice.

However, the effect of Pam-3-cys on CD86 and I-A^{b} expression was severely reduced on TLR2−/− splenic DC subsets indicating activation of DC in vivo by Pam-3-cys is TLR2-dependent. Data representative of three independent experiments.

FIG. 12. E. coli LPS and Pam-3-cys induce different classes of CD4⁺ T cell responses.

B6.PL mice reconstituted with OT-2 TCR transgenic T cells were injected i.p with class II restricted OVA peptide, OVA₃₂₃₋₃₃₉ (50 μg)+E. coli LPS (25 μg), OVA₃₂₃₋₃₃₉ (50 μg)+Pam-3-cys (50 μg) or OVA₃₂₃₋₃₃₉ alone (50 μg). Four days later, spleens were removed and clonal expansion of OVA₃₂₃₋₃₃₉ specific T cells was determined [A]. Unfractionated spleen cells were rechallenged in vitro with OVA₃₂₃₋₃₃₉ and proliferation [B] and cytokine production [C] determined. [A] Both E. coli LPS and Pam-3-cys induced clonal expansion of OVA₃₂₃₋₃₃₉ specific CD4⁺ T cells. Further, the in vitro proliferation capacity of the OVA₃₂₃₋₃₃₉ T cells was greatly enhanced in spleen cells from mice that had received the TLR ligands compared with mice that received OVA₃₂₃₋₃₃₉ alone [B]. Higher levels of IFN-γ were detected in culture supernatants from mice injected with OVA₃₂₃₋₃₃₉+E. coli LPS than those from mice which had received OVA₃₂₃₋₃₃₉+Pam-3-cys [p<0.01]. In contrast, injections of OVA₃₂₃₋₃₃₉+Pam-3-cys induced relatively higher levels of IL-4 and IL-5 [p<0.01] and similar levels of IL-13 [C]. [D] The ratio of Th1:Th2 cytokines induced by E. coli LPS is much higher than that induced by Pam-3-cys. Data representative of four independent experiments.

FIG. 13. E. coli LPS and Pam-3-cys induce different classes of CD8⁺ T cell responses. B6.PL (Thy 1.1) mice were reconstituted with OT-1 TCR transgenic T cells, and then injected i.p with class I restricted OVA peptide, OVA₂₅₇₋₂₆₄ (50 μg)+E. coli LPS (25 μg), OVA₂₅₇₋₂₆₄ (50 μg)+Pam-3-cys (50 μg) or OVA₂₅₇₋₂₆₄ alone (50 μg). Four days later, spleens were removed and clonal expansion of OVA₂₅₇264 specific T cells was determined [A]. Unfractionated spleen cells were restimulated in vitro with OVA₂₅₇₋₂₆₄ and proliferation [B] and cytokine production [C] determined. [A] Both E. coli LPS and Pam-3-cys induced clonal expansion of OVA₂₅₇₋₂₆₄ specific CD8⁺ T cells. Further, the in vitro proliferation capacity of the OVA₂₅₇₋₂₆₄ T cells was greatly enhanced in spleen cells from mice that had received the TLR ligands compared with mice that received OVA₂₅₇₋₂₆₄ alone [B]. Higher levels of IFN-γ were detected in culture supernatants from mice injected with OVA₂₅₇₋₂₆₄+E. coli LPS than those from mice which had received OVA₂₅₇₋₂₆₄+Pam-3-cys [p<0.01]. In contrast, injections of OVA₂₅₇₋₂₆₄+Pam-3-cys induced relatively higher levels of IL-4, IL-5 and IL-13 [p<0.05; C]. [D] The ratio of Th1:Th2 cytokines induced by E. coli LPS is much higher than that induced by Pam-3-cys. Data representative of three independent experiments.

FIG. 14: A model for signaling networks involved in Th1/Th2 decision making by dendritic cells. TLR 4 ligands induce potent p38 MAP kinase activation, and less ERK activation. p38 is critical for the induction of IL-12p70, and to a lesser extent IL-10. In contrast, Pam-3-cys, a TLR 2 ligand induces enhanced ERK ½ signaling, which results in the stabilization of the transcription factor c-Fos, which potently suppresses IL-12p70, and enhances IL-10, thus favoring a Th2 bias. Note that c-Fos is also likely to be stabilized by other networks. Also, note that ultimately, the responses represent a bias towards the opposite ends of the Th1/Th2 spectrum, rather than canonical Th1 or Th2 responses.

FIG. 15: A schematic diagram depicting Pam3cys.

### DETAILED DESCRIPTION OF THE INVENTION

The present invention provides methods for regulating Th immune responses. In these methods, the immune response is biased towards or against production of a Th2, Th1 or Th0 or any T regulatory cells. The method provides contacting a TLR-positive cell with an amount of a molecule effective to regulate a TLR immune response.

In accordance with the invention, the method comprises the use of agents (also referred to herein as molecules of the invention) that bias (also referred to herein as regulate) a Th (e.g., Th1, Th2, or Th0) immune response. The agent can bias a Th immune response by inducing for example, a TLR-dependent cell signaling pathway through any of TLR 1 through TLR-10, preferably, TLR2, or ERK ½ or c-fos.

As noted above, the immune system has evolved diverse types of immune responses against different pathogens. An immune response includes an immune system reaction (e.g., enhancing, stimulating, promoting, generating, producing or increasing the number of T helper cells (e.g., Th2 cells). For example, generally, viruses and bacteria stimulate the generation of T-helper (Th) cells (e.g., TH1 cells), which secrete INF-γ, and activate macrophages to produce mediators such as nitric oxide (NO) and TNF, which kill the pathogen. In contrast, parasites, such as schistosomes, generally stimulate T-helper (Th2) cells, which produce cytokines (including IL-4, IL-13, and IL-5) that induce IgE- and eosinophil-mediated destruction of the pathogen (Mosmann and Coffman, Ann. Rev. Immunol. 7:145-173, 1989; Seder and Paul, Ann. Rev. Immunol. 12:635-673, 1994; O'Garra, Immunity 8:275-283, 1993; and Murphy et al., Ann. Rev. Immunol. 18:451-494, 2000).

As used herein “regulate” and “regulating” and “modulate” and “modulating” a Th immune response(s) means an increase in biasing or decrease in biasing towards a Th immune response. Accordingly, an increase in biasing towards a Th immune response includes any of enhancing, enhancing the number and/or function of Th cells respectively, inducing, stimulating, promoting, generating or producing a Th immune response. Conversely, a decrease in biasing towards a Th immune response includes any of reducing or inhibiting a Th immune response, respectively.

T-helper cells, Th1 and Th2, can be characterized by identifying their secreted cytokines and/or by their function. For example, Th1 cells secrete IFN-gamma and/or activate macrophages to produce mediators including nitric oxide and TNF. Th2 cells secrete IL-4, IL-13 and/or IL-5. Th2 cells can induce IgE and eosinophil-mediated destruction of pathogens.

In accordance with the methods of the invention, the cell can be a cell of the immune system, e.g., a mature or immature dendritic cell (DC), such as, a monocyte derived dendritic cell, or a bone marrow precursor cell. Merely be way of example, the cell can be a myeloid DC, plasmacytoid DC, immature DC, mature DC, or mast cell. The cell can be a cell that lines the mucosal surface of a respiratory or intestinal tract. The cell may express any of or any combination of TLR 1 through TLR 10. In one embodiment, the cell expresses a TLR-2. In another embodiment, the cell expresses TLR 2 and 1, and/or expresses TLR 2 and 6. The cell or dendritic cell expresses cell antigens including CD80 and/or CD86 (e.g., immature DCs), and/or CD83 (mature DCs). The cell or dendritic cell expresses CD1a, HLA-DR and/or CD11c. The cell can be from any animal including bovine, porcine, murine, equine, canine, feline, simian, human, ovine, piscine or avian.

The molecules (e.g., agents) of the invention can be a TLR agonist or antagonist which binds or effects a TLR and induces cell signaling. The agent can be a TLR agonist or antagonist which activates the NF-KB and MAP kinase pathways in a TLR-dependent manner. The agent can be a TLR agonist or an antagonist which binds a TLR and agonizes or antagonizes, respectively, a Th immune response. An agonist increases or enhances cell signaling, or a T-helper immune response. An antagonist decreases or inhibits cell signaling, or a T-helper immune response.

The molecules (e.g., agents) of the invention can be can be naturally occurring, synthetic, or recombinantly produced, and includes, but are not limited to, any microbial or viral component or derivative thereof, including any component that is part of the structure of, or is produced by, the microbial cell or virus including, but not limited to, a cell wall, a coat protein, an extracellular protein, an intracellular protein, any toxic or non-toxic compound, a carbohydrate, a protein-carbohydrate complex, or any other component of a microbial cell or virus. The microbial cell or virus can be pathological.

In one embodiment, the molecule of the invention is an agonist (e.g., stimulator or activator) of a TLR or variant thereof, or a ligand of a TLR or its variant. The agonists include peptidoglycans (O. Takeuchi, et al., 1999 Immunity 11:443-451) or zymosans (A. Ozinsky, et al., 2000 Proc. Natl. Acad. Sci. USA 97:13766-13771). The agonists also include bacterial lipopeptides (e.g., diacylated and triacylated lipopeptides), lipoteichoic acid, lipoarabinomannan, phenol-soluble modulin, glycoinositolphospholipdis, glycolipids, porins, atypical LPS from Leptospira interrogns or Porphyromonas gingivalis, or HSP70 (for a review see K Takeda, et al., 2003 Annu. Rev. Immunol. 21:335-376). The agonists can be isolated and/or highly purified molecules. The agonists include whole molecules or fragments thereof, or naturally-occurring or synthetic. Examples include, but are not limited to, a non-toxic form of cholera toxin (Braun et al., J. Exp. Med. 189:541-552, 1999), certain forms of Candida albicans (d'Ostiani et al., J. Exp. Med. 191:1661-1674, 2000), or P. gingivalis LPS (Pulendran et al., J. Immunol. 167:5067-5076, 2001).

For example, molecules suitable for use in the methods of the invention include, but are not limited to, Pam3cys, flagellin, and E. coli LPS.

Examples of bacterial lipopeptides include bacterial cell wall lipopeptides which differ in their fatty acid chain of the N-terminal cysteines, such as diacylated and triacylated lipopeptides. For example, diacylated lipopeptides include Macrophage Activating Lipopeptide 2 kilo-Dalton from Mycoplasma fermentans or fragments thereof or synthetic analogues (e.g., MALP2, Pam2CSK4, Pam2CGNNDESNISFKEK, and Pam2CGNNDESNISFKEK-SK4). The triacylated lipopeptides include Pam3cys {S-[2,3-bis(palmitoyloxy)-(2-RS)-propyl]-N-palmitoyl-R-Cys-S-Ser-Lys4-OH)} (Takeuchi, et al., 2001 International Immunology 13:933-940).

In an embodiment, the agonist specifically effects TLR-2 or a receptor(s) bound by SEA (with respect to SEA, see MacDonald et al., J. Immunol. 167:1982-1988, 2001). Here too, the agonist can be, but is not limited to, a natural ligand, a biologically active fragment thereof, or a small or synthetic molecule. Other useful agonists may include a non-toxic form of cholera toxin (Braun et al., J. Exp. Med. 189:541-552, 1999), certain forms of Candida albicans (d'Ostiani et al., J. Exp. Med. 191:1661-1674, 2000), or Porphyromonas gingivalis LPS (Pulendran et al., J. Immunol. 167:5067-5076, 2001). These agents fail to induce IL-12(p70) and stimulate Th2-like responses.

In one embodiment, the molecule is a SEA or a component of SEA. In another embodiment, the molecule is an agonist of an ERK ½ pathway. In another embodiment, the molecule is an agonist of the ERK ½ pathway or a component of the ERK ½ pathway, such as ERK ½. In a further embodiment, the molecule is an agonist of the c-FOS pathway, or a molecule that: increases c-Fos expression; increases c-Fos RNA production; increases c-Fos RNA stability; increases c-Fos protein translation; increases c-Fos protein stability; increases post-translational modifications which will increase c-Fos activity including, but not limited to acetylation, carboxylation, glycosylation, phosphorylation, lipidation and acylation. Additional suitable molecules can be readily determined using methods known to the art to screen candidate agent molecules for the functions disclosed above.

In another embodiment, the molecule is an agonist of ERK ½ or ERK ½ pathway. In another embodiment, the molecule is an agonist of c-fos signaling, c-fos pathway, or c-fos. An example of a molecule that affects activation of ERK1/2 or ERK1/2 pathway or c-fos, c-fos pathway, or c-fos signaling is Pam3cys.

The agonists can be agonists of TLR-4 (which bias the immune response toward the Th response, e.g. TH1) include Taxol, fusion protein from Rous sarcomavirus, envelope proteins from MMTV, Hsp60 from Chlamydia pneumoniae or Hsp60 or Hsp70 from the host. Other host factors that agonize TLR-3 include the type III repeat extra domain A of fibronectin, oligosaccharides of hyaluronic acid, polysaccharide fragments of heparan sulfat, and fibrinogen. A number of synthetic compounds serve as agonists of TLR-7 (e.g., imidazoquinolin (imiquimod and R-848), loxoribine, bropirimine, and others that are structurally related to nucleic acids).

In another embodiment, the molecule is an antagonist (e.g., inhibitor or suppressor) of an intracellular pathway that impairs TLR2 signaling or activation. The antagonists include gram negative LPS, Taxol, RSV fusion protein, MMTV envelope protein, HSP60, HSP70, Type III repeat extra domain A of fibronectin, oligosaccharides of hyaluronic acid, oligosaccharide fragments of heparan sulfate, fibrinogen and flagallin (for a review see K Takeda, et al., 2003 Annu. Rev. Immunol. 21:335-376).

In an additional embodiment, the molecule is an antagonist of an intracellular pathway that impairs SEA signaling or activation. In yet one other embodiment, the molecule is an antagonist of a JNK ½ pathway. In another embodiment, the molecule is CpG DNA which activates p38 and ERK (A-K Yi, et al., 2002 The Journal of Immunolgy 168:4711-4720).

In another embodiment, the molecule is an inhibitor of ERK ½ which can inhibit maturation of dendritic cells and thus enhancing an IL12 and Th1 response. Examples of the molecule include but are not limited to PD98059 and U0126 (A. Puig-Kroger, et al., 2001 Blood 98:2175-2182).

In another embodiment, the agent or molecule inhibits c-fos signaling thus enhancing an IL12 and Th1 response. Such molecules include a DEF domain mutant of c-fos or any polypeptide having a DEF domain mutation (L. O. Murphy, et al., 2002 Nature Cell Biology 4:556-564 and Supplementary information pages 1-3), including: rat Fra-1, and Fra-2; mouse FosB, JunD, c-Jun, c-Myc, and Egr-1; and human JunB, N-Myc, and mPerl.

The present invention provides methods for regulating an immune response. In the methods of the invention an immune response is biased towards a Th immune response in a TLR-dependent manner. In one embodiment, a TLR-expressing cell is contacted with an agent that effects a bias towards a Th immune response (e.g., a Th0, Th2 or T regulatory cell immune response). For example, the agent (e.g., a natural ligand, a biologically active fragment thereof, or a small or synthetic molecule) that activatesTLR-2, ERK ½, or c-fos.

As noted above, the immune response can be regulated or modulated (e.g., increase biasing or decrease biasing toward a Th immune response) at a point in the signaling pathway downstream from receptor activation (e.g., downstream from TLR binding or downstream from TLR activation or recognition). Thus, the patient can also be treated with agents that bias the immune response by acting intracellularly on the elements of the downstream signaling pathway.

The present invention provides methods for biasing towards a Th2 immune response by inducing cell signaling (e.g., activation) of any of the MAP kinase pathways, including an ERK ½ pathway. An induced MAP kinase pathway can be characterized by an increase in the amount and/or duration of phosphorylated components of the MAP kinase pathways, including ERK ½.

In another embodiment of the methods of the invention, the agent or molecule of the invention modulates an ERK ½ MAP kinase pathway so as to regulate a TH2 immune response. In this embodiment, as an example, an agonist of a TLR (e.g., TLR-2) induces phosphorylation of ERK ½ so as to enhance a TH2 immune response.

In yet another embodiment of the methods of the invention, the molecule of the invention modulates a c-FOS pathway in the cell so as to regulate a TH2 immune response. In this embodiment, as an example, an agonist of a c-fos pathway induces expression of c-fos and/or phosphorylation of c-fos so as to enhance a TH2 immune response.

Additionally, in yet a further embodiment of the methods of the invention, the molecule of the invention modulates a Th2 immune response by affecting TLR2 or its downstream signaling pathway elements such as ERK ½ MAP kinase pathway and a c-FOS pathway. For example, the molecules of the invention can be used to modulate production or activity of IL-10 (for example increase production or upregulate of IL-10).

In one embodiment, the methods for biasing towards a Th2 immune response includes decreasing or inhibiting signaling of p38 and/or JNK pathway(s) which mediate (e.g., inhibit) IL12 production and thus biasing against a Th1 response. In another embodiment, the methods for biasing towards a Th2 immune response includes decreasing or inhibiting the amount of phosphorylated p38 and/or JNK, or decreasing or inhibiting the duration of phosphorylation of p38 and/or JNK which mediate (e.g., inhibit) IL12 production and thus biasing against a Th1 response.

The present invention also provides methods for biasing towards a Th1 immune response by inducing cell signaling (e.g., activation) of any of the MAP kinase pathways, including a p38 and/or JNK pathway. An induced p38 and/or JNK pathway can be characterized by an increase in the amount and/or duration of phosphorylated components of the MAP kinase pathways, including p38, and/or JNK.

In one embodiment, the methods for biasing towards a Th1 immune response includes decreasing or inhibiting signaling of ERK ½ and/or c-fos pathway(s). In another embodiment, the methods for biasing towards a Th1 immune response includes decreasing or inhibiting the amount of phosphorylated ERK ½ and/or c-fos, or decreasing or inhibiting the duration of phosphorylation of ERK ½ and/or c-fos.

Additionally, the invention provides methods for regulating a TH2 immune response which comprises contacting a T cell (e.g., a naïve T cell) with a TLR-positive cell (such as a DC) treated in culture with a TLR agonist (e.g., TLR-2 agonist) which activates an ERK ½ pathway and/or which activates c-fos or c-fos pathway.

Addifionally, the invention provides methods for regulating a THI immune response which comprises contacting a T cell (e.g., a naïve T cell) with a TLR-positive cell treated in culture with a TLR agonist (e.g., TLR-4 agonist) which activates a p38 pathway and/or a JNK pathway.

The present invention provides methods for treating a subject having an immune-related condition or disease (e.g., allergies, autoimmune disease, and other immune-related conditions including cancer), comprising administering to the subject any of the molecules of the invention in an amount effective to bias towards or against a Th1, Th2 or Th0 immune response. The subject can be bovine, porcine, murine, equine, canine, feline, simian, human, ovine, piscine or avian.

In one embodiment, a subject having a condition or disease associated with an exhuberant Th2 response is treated with a molecule of the invention that activates cell signaling in the subject so as to bias towards a Th1 immune response. Disease characterized by exhuberant Th2 response include, but are not limited to allergy, asthma, and chronic obstructive pulmonary disease (COPD (e.g., emphysema or chronic bronchitis).

In another embodiment, a subject having a condition or disease associated with an exhuberant Th2 response is treated with a molecule that inhibits biasing towards a Th2 immune response.

In one embodiment, a subject having a condition or disease associated with an exhuberant Th1 response is treated with a molecules of the invention that activates cell signaling in the subject so as to bias towards a Th2 immune response. Disease characterized by exhuberant Th1 response include, but are not limited to diabetes, rheumatoid arthritis, multiple sclerosis, psoriasis, and systemic lupus erythrematosis.

In another embodiment, a subject having a condition or disease associated with an exhuberant Th1 response is treated with a molecule that inhibits biasing towards a Th1 immune response.

Toll-like Receptors: The innate immune system has a series of conserved receptors, known as pattern-recognition receptors, which recognize specific pathogen-associated molecular patterns. The identification of Toll-like receptors (TLRS) that fulfill this role is an important advance in our understanding of the early events of host defense.

TLRs are type I transmembrane proteins that are evolutionarily conserved between insects and humans. So far, ten TLRs have been identified. Consistent with their function as pathogen-recognition receptors, TLRs are expressed mainly in the cell types that are involved in the first line of defense. TLRs activate signaling pathways that are similar to those that are engaged by interleukin-1 (IL-1), leading to the nuclear translocation of nuclear factor-B (NF-B) and the subsequent transcriptional activation of numerous pro-inflammatory genes.

Allergic asthma is chosen as an example of a chronic, Th2 cell-driven inflammatory disease to show how TLR agonists or antagonists might offer possibilities for therapeutic intervention.

In addition to the development of new therapies for diseases such as sepsis or disease-modifying therapies that result in immune deviation in asthma, reagents that enhance TLR-signaling pathways can be powerful adjuvants for fighting pathogens or cancer.

It has been shown that TLRs can be stimulated by endogenous ligands, such as heat-shock proteins, saturated and unsaturated fatty acids, hyaluronic-acid fragments, double-stranded DNA and surfactant protein-A.

For molecules of the invention which are proteins, nucleic acids that encode the protein-based agents of the invention (e.g., the TLR agonists and antagonists described herein) can be included in genetic constructs (e.g., plasmids, cosmids, and other vectors that transport nucleic acids) that include a nucleic acid encoding a TLR agonist or antagonist or an agent that stimulates or inhibits the associated signaling pathways in a sense or antisense orientation. The nucleic acids can be operably linked to a regulatory sequence (e.g., a promoter, enhancer, or other expression control sequence, such as a polyadenylation signal) that facilitates expression of the nucleic acid. The vector can replicate autonomously or integrate into a host genome, and can be a viral vector, such as a replication defective retrovirus, an adenovirus, or an adeno-associated virus.

The expression vector will be selected or designed depending on, for example, the type of host cell to be transformed and the level of protein expression desired. For example, when the host cells are mammalian cells, the expression vector can include viral regulatory elements, such as promoters derived from polyoma, Adenovirus 2, cytomegalovirus and Simian Virus 40. The nucleic acid inserted (i.e., the sequence to be expressed) can also be modified to encode residues that are preferentially utilized in E. coli (Wada et al., Nucleic Acids Res. 20:2111-2118, 1992). These modifications can be achieved by standard DNA synthesis techniques.

Expression vectors can be used to produce the TLR agonists or antagonists ex vivo (e.g., the proteins of the invention can be purified from expression systems known in the art) or in vivo (in, for example, whole organisms). Regardless of the manner in which it was made, once sufficiently pure, the proteins can be used as described herein. For example, one can administer the protein to a patient, use it in diagnostic or screening assays, or use it to generate antibodies.

As noted above, the methods of the invention (whether aimed at treating or preventing an autoimmune-related disorder or aimed at identifying therapeutic agents that bias the immune response) can be carried out with antibodies (i.e., immunoglobulin molecules) that specifically bind to the TLRs described herein or molecules of the invention, to components of the signaling pathways, or to the transcription factors that modulate gene expression. The methods can also be carried out with antibody fragments (e.g., antigen-binding fragments or other immunologically active portions of the antibody). Antibodies are proteins, and those of the invention can have at least one or two heavy chain variable regions (VH), and at least one or two light chain variable regions (VL). The VH and VL regions can be further subdivided into regions of hypervariability, termed “complementarity determining regions” (CDR), which are interspersed with more highly conserved “framework regions” (FR). These regions have been precisely defined (see, Kabat et al., Sequences of Proteins of Immunological Interest, Fifth Edition, U.S. Department of Health and Human Services, NIH Publication No. 91-3242, 1991 and Chothia.et al., J. Mol. Biol. 196:901-917, 1987), and specific antibodies or antibody fragments including one or more of them are within the scope of the invention.

The antibodies of the invention can also include a heavy and/or light chain constant region (constant regions typically mediate binding between the antibody and host tissues or factors, including effector cells of the immune system and the first component (C1q) of the classical complement system), and can therefore form heavy and light immunoglobulin chains, respectively. For example, the antibody can be a tetramer (two heavy and two light immunoglobulin chains, which can be connected by, for example, disulfide bonds). The heavy chain constant region includes three domains (CH1, CH2 and CH3), whereas the light chain constant region has one (CL).

An antigen-binding fragment of the invention can be: (i) a Fab fragment (i.e., a monovalent fragment consisting of the VL, VH, CL and CH1 domains); (ii) a F(ab′)₂ fragment (i.e., a bivalent fragment including two Fab fragments linked by a disulfide bond at the hinge region); (iii) a Fd fragment consisting of the VH and CH1 domains; (iv) a Fv fragment consisting of the VL and VH domains of a single arm of an antibody, (v) a dAb fragment (Ward et al., Nature 341:544-546, 1989), which consists of a VH domain; and (vi) an isolated complementarity determining region (CDR).

F(ab′)₂ fragments can be produced by pepsin digestion of the antibody molecule, and Fab fragments can be generated by reducing the disulfide bridges of F(ab′)₂ fragments. Alternatively, Fab expression libraries can be constructed (Huse et al., Science 246:1275, 1989) to allow rapid and easy identification of monoclonal Fab fragments with the desired specificity. Methods of making other antibodies and antibody fragments are known in the art. For example, although the two domains of the Fv fragment, VL and VH, are coded for by separate genes, they can be joined, using recombinant methods or a synthetic linker that enables them to be made as a single protein chain in which the VL and VH regions pair to form monovalent molecules (known as single chain Fv (scFv); see e.g., Bird et al., Science 242:423-426, 1988; Huston et al., Proc. Natl. Acad. Sci. USA 85:5879-5883, 1988; Colcher et al., Ann. NY Acad. Sci. 880:263-80, 1999; and Reiter, Clin. Cancer Res. 2:245-52, 1996).

Techniques for producing single chain antibodies are also described in U.S. Pat. Nos. 4,946,778 and 4,704,692. Such single chain antibodies are encompassed within the term “antigen-binding fragment” of an antibody. These antibody fragments are obtained using conventional techniques known to those of ordinary skill in the art, and the fragments are screened for utility in the same manner that intact antibodies are screened. Moreover, a single chain antibody can form dimers or multimers and, thereby, become a multivalent antibody having specificities for different epitopes of the same target protein.

The antibody can be a polyclonal (i.e., part of a heterogeneous population of antibody molecules derived from the sera of the immunized animals) or a monoclonal antibody (i.e., part of a homogeneous population of antibodies to a particular antigen), either of which can be recombinantly produced (e.g., produced by phage display or by combinatorial methods, as described in, e.g., U.S. Pat. No. 5,223,409; WO 92/18619; WO 91/17271; WO 92/20791; WO 92/15679; WO 93/01288; WO 92/01047; WO 92/09690; WO 90/02809; Fuchs et al., Bio/Technology 9:1370-1372, 1991; Hay et al. Human Antibody Hybridomas 3:81-85, 1992; Huse et al. Science 246:1275-1281, 1989; Griffths et al. EMBO J. 12:725-734, 1993; Hawkins et al., J. Mol Biol 226:889-896, 1992; Clackson et al. Nature 352:624-628, 1991; Gram et al., Proc. Natl. Acad. Sci. USA 89:3576-3580, 1992; Garrad et al., Bio/Technology 9:1373-1377, 1991; Hoogenboom et al. Nucl. Acids Res. 19:4133-4137, 1991; and Barbas et al., Proc. Natl. Acad. Sci. USA 88:7978-7982, 1991). In one embodiment, an antibody is made by immunizing an animal with a protein encoded by a nucleic acid of the invention (one, of course, that comprises coding sequence) or a mutant or fragment (e.g., an antigenic peptide fragment) thereof. Alternatively, an animal can be immunized with a tissue sample (e.g., a crude tissue preparation, a whole cell (living, lysed, or fractionated) or a membrane fraction). Thus, antibodies of the invention can specifically bind to a purified antigen or a tissue (e.g., a tissue section, a whole cell (living, lysed, or fractionated) or a membrane fraction).

The antibody, particularly one used therapeutically, can be a fully human antibody (e.g., an antibody made in a mouse that has been genetically engineered to produce an antibody from a human immunoglobulin sequence, such as that of a human immunoglobulin gene (the kappa, lambda, alpha (IgA1 and IgA2), gamma (IgG1, IgG2, IgG3, IgG4), delta, epsilon and mu constant region genes or the myriad immunoglobulin variable region genes). Alternatively, the antibody can be a non-human antibody (e.g., a rodent (e.g., a mouse or rat), goat, or non-human primate (e.g., monkey) antibody).

Methods of producing antibodies are well known in the art. For example, as noted above, human monoclonal antibodies can be generated in transgenic mice carrying the human immunoglobulin genes rather than those of the mouse. Splenocytes obtained from these mice (after immunization with an antigen of interest) can be used to produce hybridomas that secrete human mAbs with specific affinities for epitopes from a human protein (see, e.g., WO 91/00906, WO 91/10741; WO 92/03918; WO 92/03917; Lonberg et al., Nature 368:856-859, 1994; Green et al., Nature Genet. 7:13-21, 1994; Morrison et al. Proc. Natl. Acad. Sci. USA 81:6851-6855, 1994; Bruggeman et al., Immunol. 7:33-40, 1993; Tuaillon et al., Proc. Natl. Acad. Sci. USA 90:3720-3724, 1993; and Bruggeman et al., Eur. J. Immunol 21:1323-1326, 1991).

The antibody can also be one in which the variable region, or a portion thereof (e.g., a CDR), is generated in a non-human organism (e.g., a rat or mouse). Thus, the invention encompasses chimeric, CDR-grafted, and humanized antibodies and antibodies that are generated in a non-human organism and then modified (in, e.g., the variable framework or constant region) to decrease antigenicity in a human. Chimeric antibodies (i.e., antibodies in which different portions are derived from different animal species (e.g., the variable region of a murine mAb and the constant region of a human immunoglobulin) can be produced by recombinant techniques known in the art. For example, a gene encoding the Fc constant region of a murine (or other species) monoclonal antibody molecule can be digested with restriction enzymes to remove the region encoding the murine Fc, and the equivalent portion of a gene encoding a human Fc constant region can be substituted therefore (see European Patent Application Nos. 125,023; 184,187; 171,496; and 173,494; see also WO 86/01533; U.S. Pat. No. 4,816,567; Better et al., Science 240:1041-1043, 1988; Liu et al., Proc. Natl. Acad. Sci. USA 84:3439-3443, 1987; Liu et al., J. Immunol. 139:3521-3526, 1987; Sun et al., Proc. Natl. Acad. Sci. USA 84:214-218, 1987; Nishimura et al., Cancer Res. 47:999-1005, 1987; Wood et al., Nature 314:446-449, 1985; Shaw et al., J. Natl. Cancer Inst. 80:1553-1559, 1988; Morrison et al., Proc. Natl. Acad. Sci. USA 81:6851, 1984; Neuberger et al., Nature 312:604, 1984; and Takeda et al., Nature 314:452, 1984).

The nucleic acids, proteins, cells, and antibodies described herein can be used in, for example, screening assays, therapeutic or prophylactic methods of treatment, or predictive medicine (e.g., diagnostic and prognostic assays, including those used to monitor clinical trials, and pharmacogenetics).

Screening Assays. The invention provides methods (or “screening assays”) for identifying agents (or “test compounds” that bind to or otherwise modulate (i.e., stimulate or inhibit) the expression or activity of a TLR described herein or a component of its effector pathway. An agent may, for example, be a small molecule such as a peptide, peptidomimetic (e.g., a peptoid), an amino acid or an analog thereof, a polynucleotide or an analog thereof, a nucleotide or an analog thereof, or an organic or inorganic compound (e.g., a heteroorganic or organometallic compound) having a molecular weight less than about 10,000 (e.g., about 5,000, 1,000, or 500) grams per mole and salts, esters, and other pharmaceutically acceptable forms of such compounds.

Libraries of compounds may be presented in solution (see, e.g., Houghten, Biotechniques 13:412-421, 1992), or on beads (Lam, Nature 354:82-84, 1991), chips (Fodor, Nature 364:555-556, 1993), bacteria or spores (U.S. Pat. No. 5,223,409), plasmids (Cull et al., Proc Natl Acad Sci USA 89:1865-1869, 1992) or on phage (Scott and Smith, Science 249:386-390, 1990; Devlin, Science 249:404-406, 1990; Cwirla et al., Proc. Natl. Acad. Sci. USA 87:6378-6382, 1990; Felici, J. Mol. Biol. 222:301-310, 1991; and U.S. Pat. No. 5,223,409).

The screening assay can be a cell-based assay, and the cell can be a dendritic cell or other cell that expresses a TLR. The cell used can be a mammalian cell, including a cell obtained from a human or from a human cell line. The screening assays can also be cell-free assays (i.e., soluble or membrane-bound forms of the TLRs). The basic protocol is the same as that for a cell-based assay in that, in either case, one must contact a TLR-positive cell with an agent of interest (for a sufficient time and under appropriate (e.g., physiological) conditions to allow any potential interaction to occur) and then determine whether the agent binds the TLR-positive cell or otherwise modulates an ERK ½ pathway, and/or a c-fos pathway. The TLR-positive cell, so contacted can be used to induce T-cell proliferation and/or induce T-cell development towards or against a TH2 cell. Those of ordinary skill in the art will, however, appreciate that there are differences between cell-based and cell-free assays.

The following examples are presented to illustrate the present invention and to assist one of ordinary skill in making and using the same. The examples are not intended in any way to otherwise limit the scope of the invention.

### EXAMPLES

### Example 1

The studies that follow demonstrate that the response of T-helper (Th) cells to a given immunogen varies depending, at least in part, on which receptor (e.g., which TLR) the immunogen activates. Accordingly, one can bias the immune response by activating or inhibiting particular receptors, including TLRs.

Some of our key findings, as well as our conclusions regarding the underlying cellular mechanisms, can be summarized as follows. Two agents from the bacterial pathogen E. coli, lipopolysaccharide (LPS) and flagellin, trigger TLR4 and TLR5, respectively. Activation of these receptors instructs DCs to stimulate polarized Th1 responses via the production of IL-12(p70), which depends on the phosphorylation of p38 and JNK1/2 MAP kinases. In contrast, the synthetic TLR2 agonist Pam3cys, and schistosome egg antigens (SEA): (i) do not induce IL-12(p70); (ii) stimulate sustained duration of ERK1/2 phosphorylation, which stabilizes the early-growth transcription factor c-Fos, a suppressor of IL-12; (iii) and elicit Th0/Th2 responses. Thus, different receptor agonists, which differentially activate intracellular signaling pathways, stimulate expression of distinct cytokines from DCs and influence Th cell responses.

Reagents: Highly purified E. coli LPS was generated in the laboratory of Dr. Thomas Van Dyke, as described in Pulendran et al. (J. Immunol. 167:5067-5076, 2001). Highly purified flagellin was generated in the laboratory of Dr. Andrew Gewirtz, as described in McSorley et al. (J. Immunol. 169:3914-3919, 2002). Soluble S. mansoni egg antigens (SEA) were purified in the laboratory of Dr. Barbara Doughty, as described in Moyes et al. (Parasite Immunol. 18:625-633, 1996). Pam3cys (e.g., see FIG. 15)

Isolation and culture of human monocyte-derived DCs: CD14⁺ monocytes were enriched from peripheral blood mononuclear cells (PBMCs), using an enrichment step, and cultured in six well plates (1×10⁶ cells/well) for six days with recombinant Human GM-CSF at 100 ng/ml (PeproTech, NJ) and recombinant Human IL-4 at 20 ng/ml (PeproTech, NJ). At day six, the cultures consisted uniformly of CD1a⁺CD14⁻, HLA-DR⁺ CD11c⁺ cells, which were negative for CD83, the human DC maturation marker, and expressed intermediate levels of such costimulatory molecules as CD86 (Rissoan et al., Science 283:1183-1186, 1999). On day six, the immature DCs were pulsed with E. coli LPS (1 mg/ml), flagellin (0.5 mg/ml), Pam3cys (20 mg/ml), or SEA (100 mg/ml) for 48 hours.

DC phenotype: The phenotype of DCs was determined by flow cytometry using a Facscalibur (BD Pharmingen, CA). Briefly, gated CD1a⁺CD14⁻, CD11c⁺ HLA-DR⁺ DCs were analyzed for the expression of CD80, CD86, CD83 and CD40. All antibodies, including the PE-labeled isotype controls were purchased from BD Pharmingen (La Jolla, Calif.).

Cytokine Production by DCs: The cytokines secreted by DCs cultured with various stimuli were measured by ELISA (using kits from BD Pharmingen, La Jolla, Calif.). For inhibition studies, DCs were incubated with commercially available inhibitors of p38 (SB203580 (Calbiochem, CA); Yi et al., J. Immunol. 168:4711-4720, 2002; Yi et al., International Immunol. 13:1391-1404, 2002; Park et al., Science 297:2048-2051, 2002), ERK1/2 (UO126; a specific inhibitor of the upstream activators of MAP-kinase kinase 1 & 2 (MEK 1 & 2) (see Yi et al., Yi et al., and Park et al. supra), or JNK1/2 (Park et al., supra), for one hour, before adding the stimuli.

Evaluation of MAP-kinase intracellular signaling pathways: Evaluation of MAP kinase signaling was done using the Western Blot technique or commercially available ELISA kits (BioSource; according to manufacturer's instructions). Briefly, Day 6, immature, human monocyte-derived DCs (2×10⁶) were cultured for 15 minutes, 1 hour, or 4 hours with various stimuli. For Western blot analysis, the same number and type of DCs were examined as were examined by ELISA. Cellular extracts were prepared as described in the Biosourse ELISA Kit, and total protein (80-100 mg) was resolved on 10% SDS-PAGE gels and transferred to ImmunoBlot PVDF membranes (Bio-Rad). Blotting was performed with anti-phospho-SAPK/JNK or total SAPK/JNK (New England Biolabs). Protein bands were visualized with secondary HRP-conjugated antibody and the SuperSignal West Pico Chemiluminescent Substrate (Pierce).

Evaluation of c-Fos expression in DCs: We examined the expression of total c-Fos and phosphorylated c-Fos (Phos. c-Fos), in DCs by FACS using antibodies directed against the two different forms of c-Fos (Murphy et al., Nature Cell Biol. 4:556-564, 2002). Day 6, immature, human monocyte derived DCs (1×10⁶) were stimulated for 15 minutes, 1 hour, or 4 hours with various stimuli at 37° C. Cells were then fixed in 2% paraformaldehyde (10% ultrapure EM grade; Polysciences, Warrington, Pa.) for 10 minutes at 37° C. We rendered the cells permeable by incubating them with freshly prepared, ice-cold methanol (90%) for 30 minutes. The cells were then washed twice in staining buffer (3% FCS in PBS), labeled with a 1:100 dilution of c-fos antibody (Santacruz Biotechnology) or phospho c-Fos antibody (by 30 minute incubations on ice), washed in staining buffer, and labeled an FITC-labeled goat anti-rabbit Ig (BD Biosciences). Flow cytometry was carried out with FACScaliber.

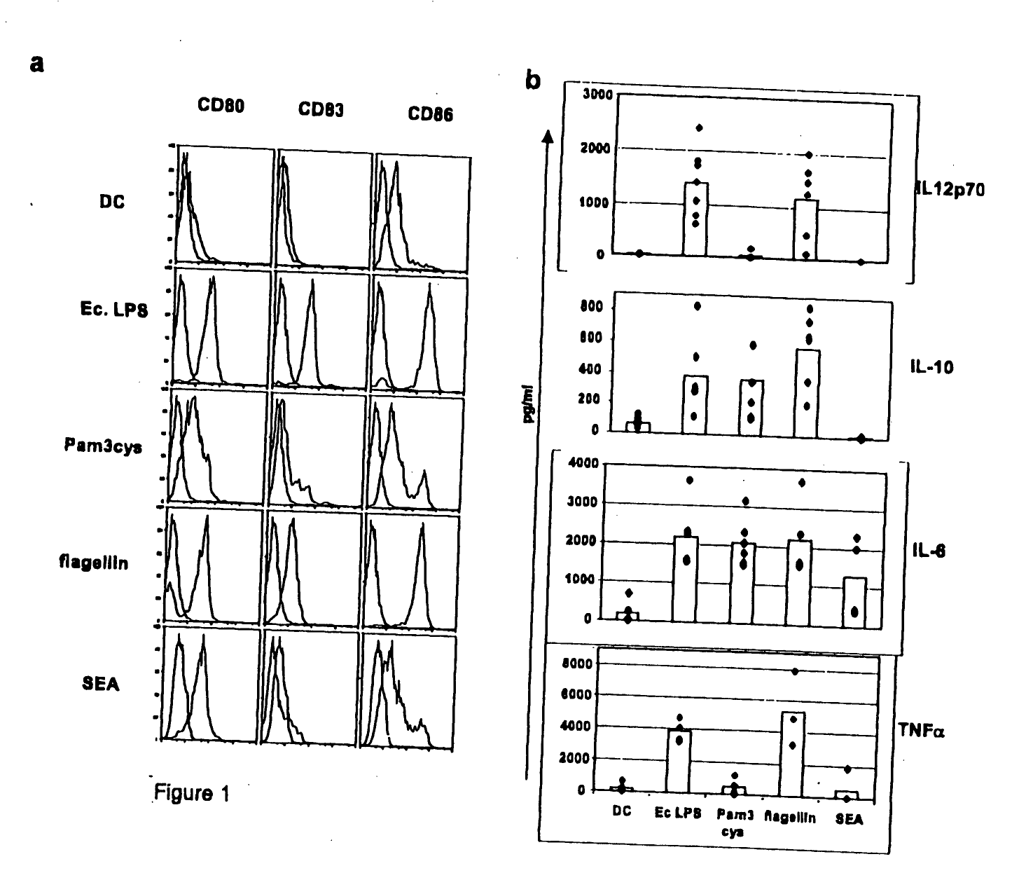

Different TLR-agonists elicit distinct responses from human monocyte-derived DCs:To study the direct effects of different TLR agonists on the functional responses of DCs, uncommitted, immature monocyte-derived DCs were cultured in the presence of pre-determined concentrations of highly purified E. coli LPS (a TLR4 stimulus), Pam3cys (a TLR2 stimulus), highly purified flagellin (TLR5 stimulus) and SEA, a classic Th2 stimulus (the cell surface receptor that mediates the response to SEA is unknown). As controls, DCs were cultured in the absence of any stimulus. As shown in FIG. 1A, all stimuli induced the maturation of DCs within 48 hours, as evidenced by up-regulation of the costimulatory molecules, CD80 and CD86. Most stimuli also induced the expression of the DC maturation marker, CD83. In the case of Pam3cys and SEA, the degree of maturation induced varied among different donors, and was weaker than that induced by E. coli LPS or flagellin, as judged by the lower levels of CD83. Notably, while all stimuli induced CD80 to similar levels, both Pam3cys and SEA induced much lower levels of CD86.

We next examined cytokine secretion from DCs in response to various doses of the different stimuli. Based on this analysis, in all further experiments, we chose doses that triggered equivalent levels of IL-6 production at 48 hours (FIG. 1B). Interestingly, there were striking differences in the ability of different stimuli to induce IL-12(p70). E. coli LPS and flagellin induced approximately 1000 pg/ml of IL-12(p70), but Pam3cys and SEA induced little or no IL-12(p70) (FIG. 1B). As indicated, the absolute amounts of cytokine secreted varied significantly from donor to donor, but the relative levels of the cytokines induced by different stimuli was consistent. IL-10, a regulatory cytokine that dampens both Th1 and Th2 responses in humans (Hemmi et al., Nature 408:740-745, 2000), was induced by E. coli LPS, flagellin and Pam3cys (FIG. 1B). The pro-inflammatory cytokine, TNF-α was strongly induced by Ec.LPS and flagellin, but induced at much weaker levels by Pam3cys and SEA. Taken together, these data suggest that the different stimuli induce very distinct cytokine profiles from DCs. In particular, Pam3cys and SEA induce little or no IL-12(p70), relative to the TLR4 and TLR5 ligands.

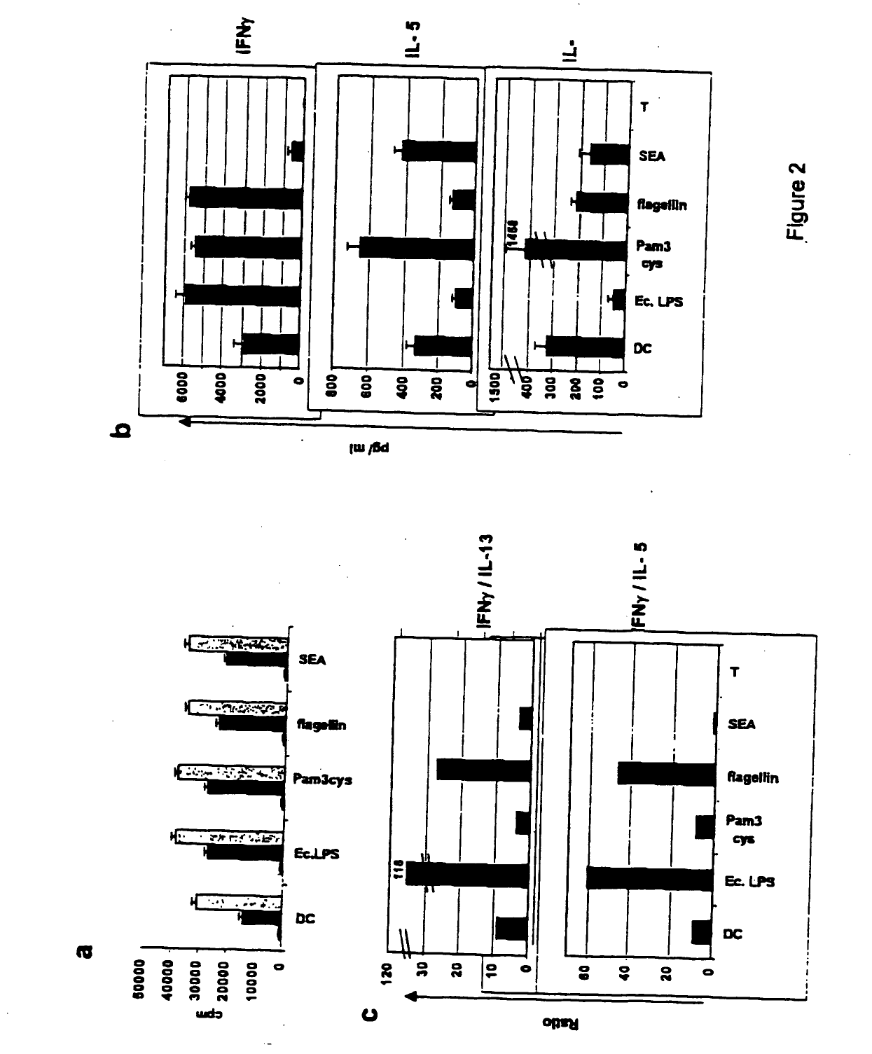

E. coli LPS and flagellin induce Th1 responses via an IL-12 dependent mechanism, but Pam3cys and SEA induce Th2/Th0 responses. Given these differences in cytokine secretion, we hypothesized that DCs stimulated with various receptor agonists could induce different types of Th responses. DCs cultured for 48 hours with the various stimuli were washed and cultured, at graded doses, with naïve, allogeneic, CD4⁺ CD45RA⁺ CD45RO⁻ T cells. After five days, the cultures were pulsed with tritiated thymidine (³[H]) for 12 hours to measure the proliferation of T cells. As seen in FIG. 2A, in all cases, DCs induced efficient T cell proliferation. The Th cytokines secreted in culture were determined by cytokine ELISA (FIG. 2B). DCs cultured in the absence of any stimuli induced about 3000 pg/ml of the Thi cytokine IFNγ, and 300-400 pg/ml of the Th2 cytokines IL-5 and IL-13 (this profile being consistent with a Th0 response). However, DCs stimulated with E. coli LPS or flagellin induced approximately 6000 pg/ml of IFNγ and much lower levels of IL-5 and IL-13, a typical Th1 profile (this finding is consistent with the high levels of IL-12(p70) induced by these stimuli (FIG. 1A)). In contrast, DCs stimulated with Pam3cys or SEA induced Th0 or Th2 responses. In particular, SEA induced a Th2 response, with less than 3000 pg/ml of IFNγ (less than uncommitted DCs), but 400 pg/ml of IL-5, and 150 pg/ml of IL-13. Pam3cys induced a typical Th0 response, with high levels of IFNγ and IL-5 and very high levels of IL-13 (1458 pg/ml). Interestingly, IL-4 could not be detected in any of the cultures, even after restimulation of the T cells with anti-CD3⁺ anti-CD28, or PMA⁺ ionomycin. Nevertheless, these data suggest that TLR4 and TLR5 ligands induce uncommitted DCs to adopt a Th-1 inducing mode, but Pam3cys and SEA induce DCs to adopt a Th0 or Th2 inducing mode. This is underscored by the ratios of IFNγ/IL-5 or IFNγ/IL-13, which reflect the Th1/Th2 balance (FIG. 2c). While E. coli LPS and flagellin favor Th1 responses, Pam3cys and SEA clearly tilt the balance towards Th2 responses (FIG. 2b).

To determine whether E. coli LPS and flagellin induce Th1 responses via an IL-12(p70) mechanism, we blocked the activity of IL-12(p70) in the DC-T cell cultures using a neutralizing antibody against the IL-12 receptor β chain (Ma et al., Ann. Rev. Immunol. 79:55-92, 2001). In all cases, the induction of IFNγ was diminished or abrogated. Based on these results, we concluded that TLR4 and TLR5 ligands induce Th1 responses via IL-12(p70), but TLR2 ligands or SEA induce Th2/Th0 responses, likely via a default mechanism that fails to induce IL-12(p70).

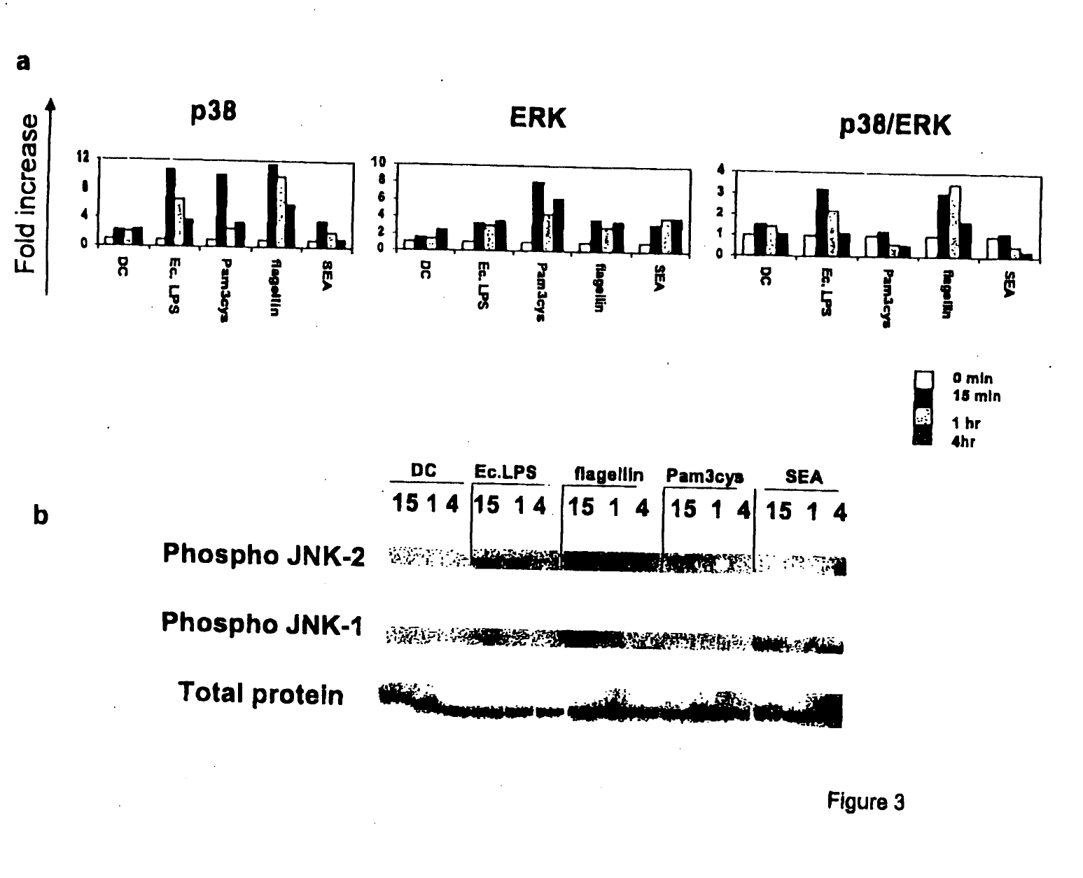

Pam3cys and SEA induce enhanced duration of ERK1/2 signaling: To gain insights into the potential intracellular signaling mechanisms that may mediate the different DC responses, we focused on the MAP-kinase signaling pathway, one of the most ancient signal transduction pathway in mammalian cells (Dong et al., Ann. Rev. Immunol. 20:55-72, 2002; Chang and Karin, Nature 410:37-40, 2001; and Davis, Cell 103:239-252, 2000). MAP-kinases consist of three major groups: (1) p38 MAP kinases, (2) the extracellular signal-regulated protein kinases (ERK1 and 2), and (3) the c-Jun NH₂-terminal kinases (JNK 1 and 2) (Dong et al., supra; Chang and Karin, supra; Davis, supra). Previous reports indicate a critical role for MAP-kinases in regulating Th1/Th2 balance in T cells (Dong et al., supra; Chang and Karin, supra; Davis, supra), and emerging evidence suggests a role for these proteins in regulating cytokine production from DCs (Davis, supra, Yi et al., supra, Yi et al., supra, and Park et al., supra). We therefore sought to determine the phosphorylation of p38, ERK1/2 and JNK1/2 in DCs stimulated with various stimuli. As shown in FIG. 3A, we found differences in the magnitude and duration of phosphorylation of the MAP kinases induced by the different stimuli. E. coli LPS and flagellin induced enhanced phosphorylation of p38 MAP kinase, which peaked at 15 minutes, and was sustained at levels well above baseline, even at 4 hours. In contrast, Pam3cys resulted in a rapid burst of p38 phosphorylation at 15 minutes, which rapidly declined to near baseline levels by 1 hour (FIG. 3a). SEA induced very little phosphorylation of p38. With regard to ERK1/2 phosphorylation, Pam3cys induced a much higher magnitude and duration of phosphorylation (which was sustained even at 4 hours), compared to E. coli LPS and flagellin. SEA also induced ERK1/2 phosphorylation, which, while weaker than that induced by Pam3cys, was sustained at 4 hours at levels significantly higher than background levels. These differences in the magnitude and duration of p38 and ERK1/2 phosphorylation were underscored by the ratios of p38:ERK, which were strikingly different in DCs stimulated by the different stimuli. DCs stimulated with E. coli LPS and flagellin expressed much higher ratios of p38:ERK1/2 compared to Pam3cys and SEA (FIG. 3a).

We also examined the phosphorylation of JNK1 and 2 induced by the various stimuli. As shown in FIG. 3b, stimulation with E. coli LPS and flagellin induced higher levels of JNK1 and 2 than stimulation with Pam3cys and SEA. Therefore, distinct TLR ligands induce differences in the magnitude and duration of signaling of MAP-kinases in DCs.

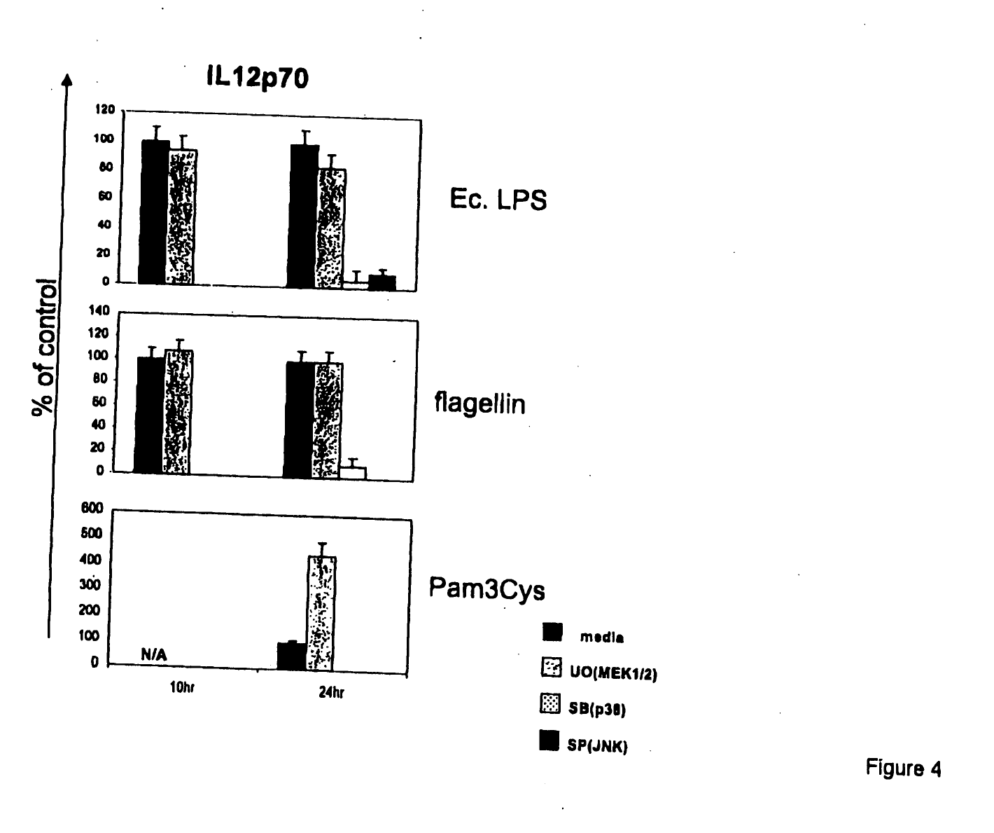

Induction of IL-12(p70) is enhanced by p38 and JNK1/2 signaling, and suppressed by ERK1/2 signaling. To examine the roles p38, JNK1/2 and ERK1/2 play in IL-12(p70) induction by DCs, we used the well characterized, highly selective, synthetic inhibitors of p38 (SB203580), ERK1/2 (UO126, a specific inhibitor of the upstream activators of MAP-kinase kinase 1 and 2 (MEK 1 and 2), or JNK1/2 (SP600125 (see the references cited above). Blocking p38 or JNK1/2, but not ERK1/2 activity, largely abrogates IL-12(p70) production induced by E. coli LPS and flagellin (FIG. 4). Interestingly, blocking ERK1/2 activity significantly enhances IL-12(p70) production induced by Pam3cys, suggesting an important role for ERK1/2 in the regulation of IL-12(p70) production. In the case of SEA, blocking ERK1/2 did not result in consistent increases in IL-12(p70). This suggests that additional mechanisms regulate the suppression of IL-12(p70) by SEA. Based on these data, we concluded that TLR4 and TLR5 agonists preferentially induce IL-12(p70) via a mechanism involving p38 and JNK1/2 phosphorylation. In contrast, Pam3cys and SEA induce enhanced duration of ERK1/2 phosphorylation, a negative regulator of IL-12(p70).

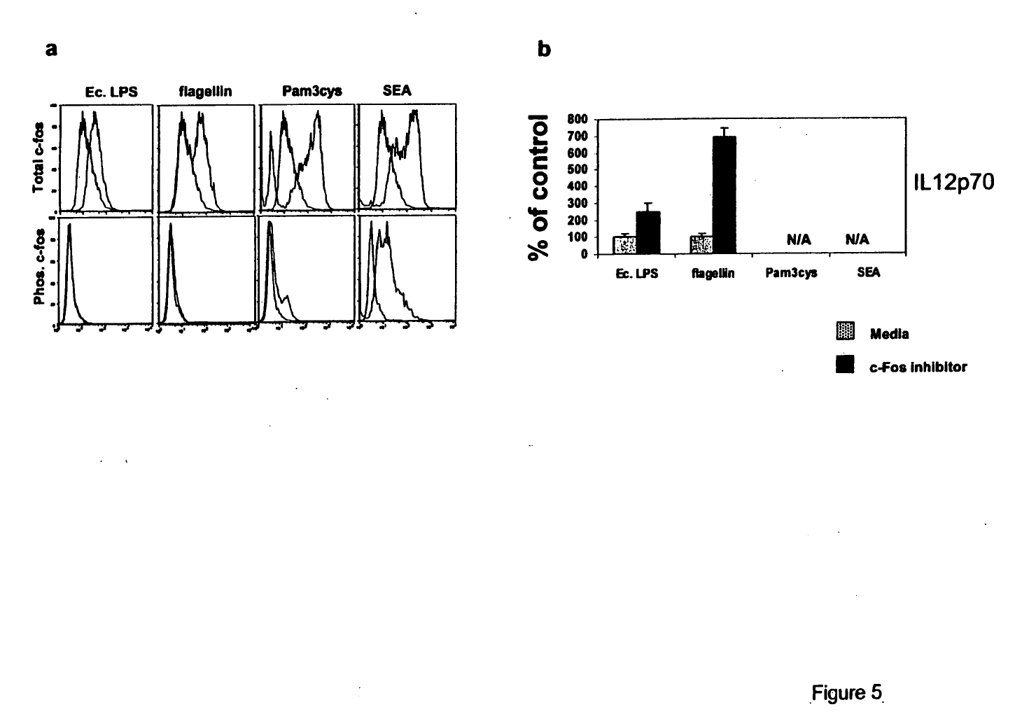

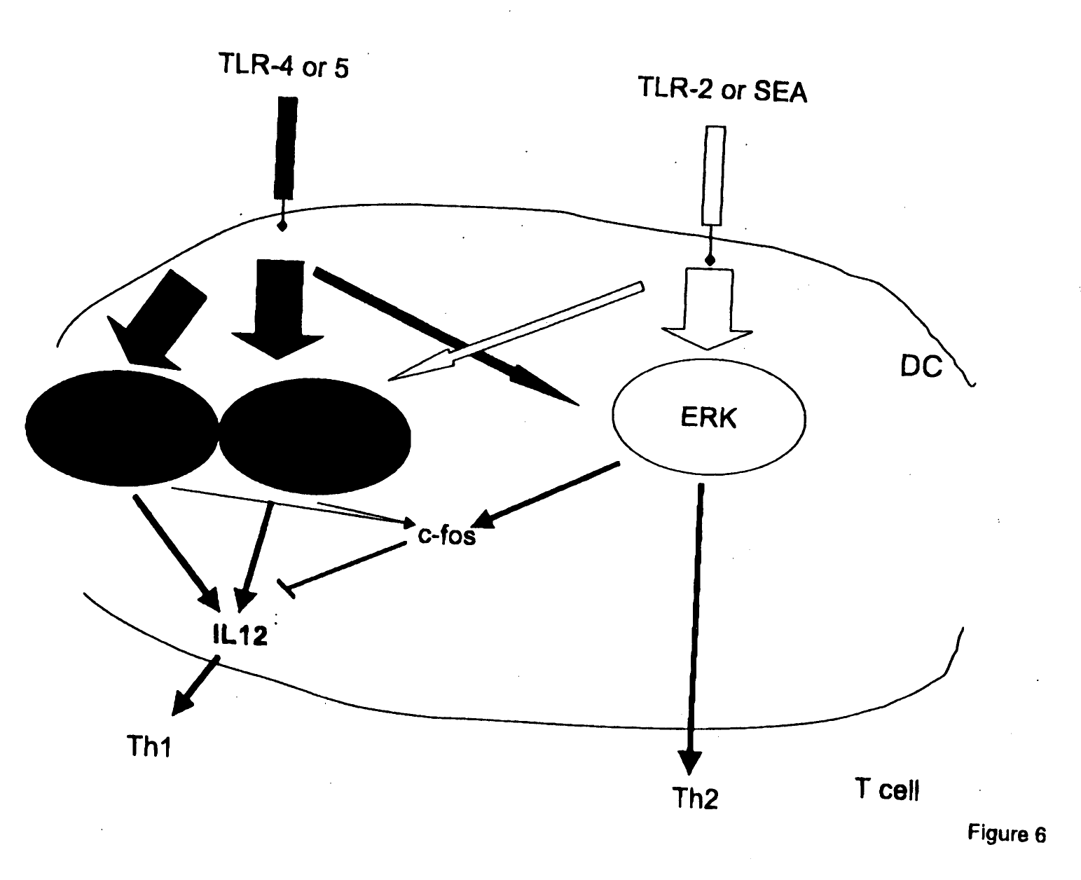

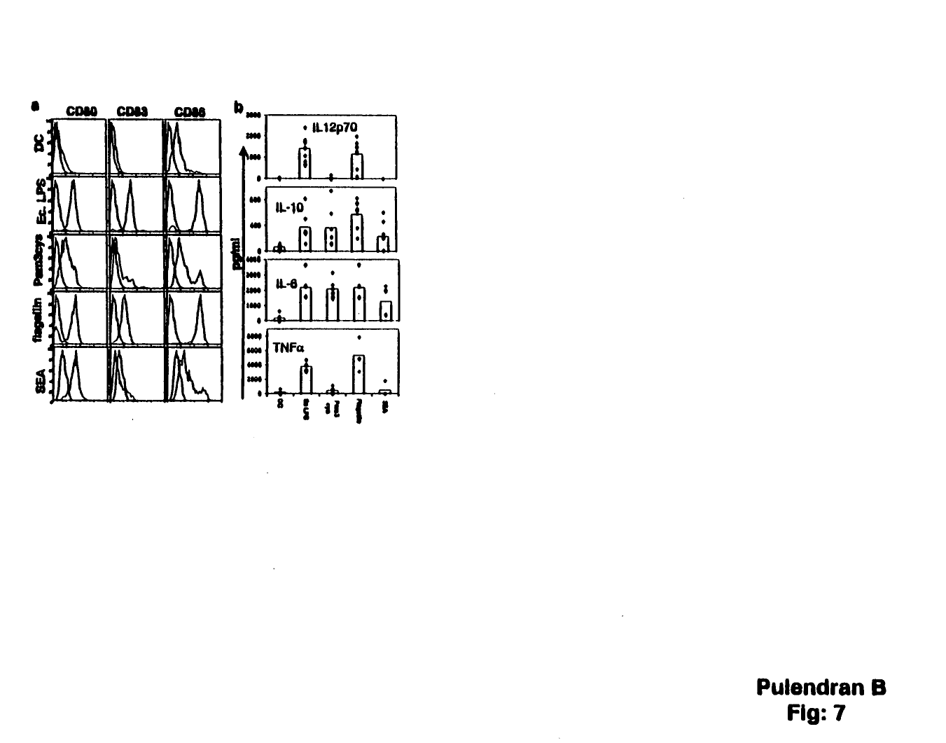

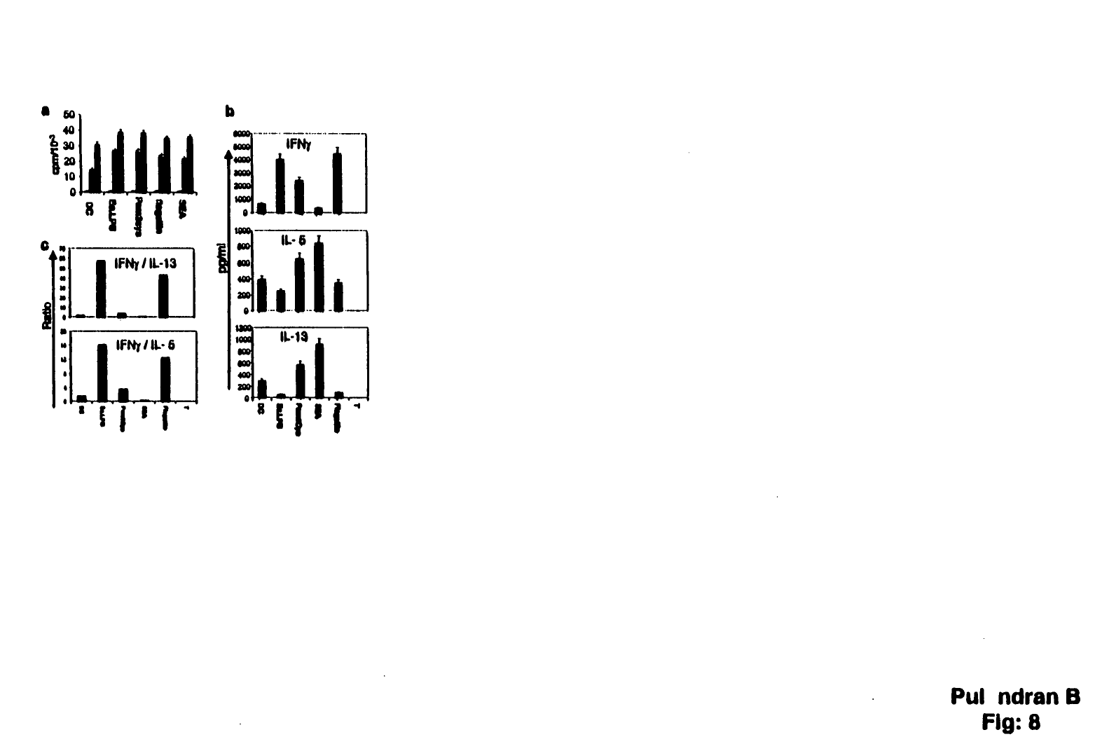

Pam3cys and SEA induce stabilization of immediate early gene product c-Fos, which regulates the production of IL-12(p70). We also asked how the enhanced duration of ERK1/2 signaling induced by Pam3cys and SEA results in suppression of IL-12(p70). Recent work carried out in a fibroblast cell line suggests that sustained ERK signaling results in the phosphorylation and stabilization of the immediate early gene product c-Fos (Murphy et al., Nature Cell Biol. 4:556-564, 2002). Furthermore, phosphorylation of c-Fos in the C-terminus appears to prime the protein for additional phosphorylation by exposing a novel DEF domain, (an FXYP-like sequence (Jones et al., J Leukoc. Biol. 6:1036-1044, 2001), which acts as an ERK binding site (Murphy et al.). Thus, we determined the kinetics and magnitude of expression of both total c-Fos and phosphorylated c-Fos (Phos. c-Fos) in DCs stimulated with the various stimuli, using antibodies directed against the two different forms of c-Fos. c-Fos expression peaked after two hours of stimulation. At this time point, the level of expression of total c-Fos, (as assessed by the mean-flourescense intensity of staining) and the fraction of cells expressing c-Fos in DCs stimulated by Pam3cys or SEA is much greater than in DCs stimulated with E. coli LPS or flagellin (FIG. 5a). Consistent with this, the more stable, phosphorylated c-Fos, was not expressed in DCs stimulated with flagellin and E. coli LPS, but was expressed at significant levels in DCs stimulated with Pam3cys and SEA. Furthermore, c-Fos expression was maintained, even at 4 hours, in DCs stimulated with Pam3cys or SEA, but not with E. coli LPS or flagellin. Therefore, stimulation of DCs by Pam3cys and SEA, which induce sustained duration of ERK1/2 signaling, result in the phosphorylation and stabilization of c-Fos.

To study the role, if any, c-Fos plays in the regulation of IL-12(p70), we used a synthetic peptide that encompasses the DEF domain and thus competitively inhibits ERK-regulated phosphorylation of c-Fos (Jacobs et al., Genes Dev. 13163-175, 1999). Incubation of DCs with various concentrations of this inhibitor peptide, before stimulation with E. coli LPS or flagellin, induced a striking increase in IL-12(p70) production (FIG. 5b). In contrast, IL-12(p70) was not consistently enhanced in response to Pam3cys and SEA (FIG. 5b), even when the concentration of peptide was enhanced to several times greater than its IC₅₀. This suggests that additional mechanisms are involved in the regulation of IL-12(p70) by Pam3cys and SEA.

### Example 2

Toll-Like Receptor Ligands Cause Dendritic Cells to Induce T-Helper Cell Responses

Dendritic cells (DCs) are pivotal in determining the class of an adaptive immune response.

However the molecular mechanisms within DCs, that determine this decision-making process, are unknown. This example demonstrates that distinct Toll-like receptor (TLR) ligands instruct human DCs to induce distinct T-helper cell (Th) responses, by differentially modulating MAP-kinase signaling.

Materials & Methods

Reagents: Highly pure Ec.LPS (Pulendran B., et al., 2001, J. Immunol., 167: 5067) and flagellin (McSorley, S. J., B. D. Ehst, Y. Yu, A. T. Gewirtz, 2002, J Immunol, 169:3914) were provided by Drs. Van Dyke and Gewirtz, respectively. SEA was purified by Dr. Barbara Doughty (Moyes, R. B., Alves-Oliveira, L. F., Parra, J. C., Gazzinelli, G., Doughty, B. L. 1996. Parasite Immunol 18:625) Pam-3-cys (Akira, S., K. Takeda, T. Kaisho. 2001. Nature Immunol. 2: 675) was purchased from Dr. Jung, at Institute of Organic Chemistry, University of Tuebingen.

Isolation and culture of human monocyte-derived DCs: CD14⁺ monocytes were enriched from peripheral blood mononuclear cells, and cultured for 6 days with recombinant human GM-CSF at 100 ng/ml (PeproTech, NJ) plus recombinant human IL-4, at 20 ng/ml (PeproTech). At day 6, the cultures consisted uniformly of CD1a⁺ CD14⁻, HLA-DR⁺ CD11c⁺ cells, which were negative for CD83. These immature DCs were pulsed with Ec.LPS (1 μg/ml), flagellin (0.5 μg/ml), Pam3cys (20 μg/ml), or SEA (100 μg/ml) for 48 h.

DC phenotype: This was determined by flow cytometry using a Facscalibur (BD Pharmingen, CA). Briefly, gated CD1a⁺CD14⁻, C11c+ HLA-DR⁺DCs were analyzed for the expression of CD80, CD86, CD83 and CD40 (BD Pharmingen, La Jolla, Calif.).

Cytokine production by DCs: This was measured by ELISA (BD Pharmingen, CA). For inhibition studies, DCs were incubated with commercially available [Calbiochem, CA], inhibitors of p38 (SB203580, (Yi, A. K., J. G. Yoon, S. J. Yeo, S. C. Hong, B. K. English, A. M. Krieg. 2002. J. Immunol. 168: 4711)), ERK1/2 (UO126—a specific inhibitor of MEK 1 & 2 (Yi, A. K., J. G. Yoon, S. J. Yeo, S. C. Hong, B. K. English, A. M. Krieg. 2002. J. Immunol. 168: 4711)) or JNK1/2 (Park, J. M., F. R. Greten, Z. W. Li, M. Karin. 2002. Science 297:2048) for 1 hr, before adding the stimuli.

DC-T cell cultures: At day 6, immature DCs were pulsed with Ec.LPS (11 μg/ml), flagellin (0.5 μg/ml), Pam3cys (20 μg/ml), or SEA (100 μg/ml) for 48 h, then washed and cultured at graded doses, with 10⁵ FACS sorted, naïve CD4+ CD45RA+ CD45RO− T cells. After 5 days, T-cell proliferation was assessed by overnight [³H] thymidine labeling. The secretion of Th1 and Th2 cytokines was assessed by ELISA.

Evaluation of MAP-kinase signaling: This was done using by western blotting or commercially available ELISA kits (BioSourse). Briefly, Day 6, immature, human monocyte-derived DCs (2×10⁶) were cultured for the indicated times, with various stimuli.

ELISA assays were performed according to manufacturer instructions. For western blotting, cellular extracts were prepared, as described in Biosource ELISA Kit), and total protein (80-100 μg) was resolved on 10% SDS-PAGE gels and transferred to Immuno Blot PVDF membranes (Bio-Rad). Blotting was performed with anti phospho-SAPK/JNK, p38 or ERK1/2 or anti-total SAPK/JNK, p38 or ERK1/2 antibodies (New England Biolabs). Bands were visualized with secondary HRP-conjugated antibody and the SuperSignal West Pico Chemiluminescent Substrate (Pierce).

Flow cytometric evaluation of c-Fos and phospho-ERK expression in DCs: The expression of total c-Fos, phosphorylated c-Fos (Phos. c-Fos), or phospho ERK in DCs was determined by FACS using antibodies directed against the two different forms of c-Fos (Murphy, L. O., S. Smith, R. H. Chen, D. C. Fingar, J. Blenis. 2002. Nat Cell Biol. 4:556). Day 6, human monocyte derived DCs were stimulated for 0.25 hr, 1 hr, and 4 hrs with various stimuli. Cells were then fixed in 2% paraformaldehyde (10% ultrapure EM grade; Polysciences, Warrington, Pa.) for 10 min at 37° C. After washing, permeabilization was done with freshly prepared 90% ice cold methanol for 30 min on ice. Then the cells were washed twice in staining buffer (3% FCS in PBS), and labeled with 1:100 dilution of c-fos antibody (Santacruz Biotechnology) and phospho c-Fos, or phospho ERK (BD Pharmingen) for 30 min on ice, then washed in staining buffer and labeled using FITC labeled goat anti rabbit Ig (BD biosciences). Flow cytometry was done on FACScaliber.

Si RNA: 5 target sequences of 21 nucleotide c-fos siRNA was selected from the web site (http://www.ambion.com/techlib/misc/siRNA_finder.html) for silencing the gene. The transcription of siRNA and transfection in dendritic cells was done as per instructions from Ambion. Briefly cells were transfected by 20 nM si RNA using siPORT lipid transfection protocol, after 6-7 hrs of transfection cells were stimulated by stimili for 40 hrs and cytokine secretion was assayed by ELISA kit (BD Bioscience).

Results

Different TLR-Agonists Elicit Distinct Responses from Human Monocyte-Derived DCs

To study the direct effects of different TLR agonists on the functional responses of DCs, uncommitted, immature human monocyte-derived DCs were cultured in the presence of pre-determined concentrations of highly purified E. coli LPS (Ec LPS-TLR4 stimulus), the synthetic TLR2 agonist Pam3cys, and highly purified flagellin (TLR5 stimulus). In addition, DCs were also cultured with SEA, a classic Th2 stimulus. Although the receptor through which SEA signals is not definitively known, SEA was used as a positive control to induce Th2 responses. As a negative control, DCs were cultured in the absence of any stimulus. As shown in FIG. 7a, all stimuli induced the maturation of DCs within 48 h, evidenced by the up-regulation of the costimulatory molecules, CD80 and CD86, although the induction of CD86 by Pam-3-cys and SEA was weaker than that by LPS and flagellin. CD80 induction by Pam-3-cys was also weaker. Most stimuli also induced the expression of the DC maturation marker, CD83. In the case of Pam3cys and SEA, the degree of maturation induced varied among different donors, and was weaker than that induced by Ec.LPS or flagellin, as judged by the lower levels of CD83. Notably, while all stimuli induced significant expression of the costimulatory molecules CD80 and CD86, both Pam3cys and SEA induced much lower levels of CD86.

The secretion of cytokines, by DCs in response to various doses of the different stimuli was examined. Based on this analysis, in all further experiments, doses of stimuli were chosen which triggered equivalent levels of IL-6 production at 48 h, as indicated in FIG. 7b.

Interestingly, there were striking differences in the levels of the Th1 inducing cytokine IL-12(p70), induced by the different stimuli. Ec.LPS and flagellin induced approximately 1000 pg/ml of IL-12(p70), but Pam3cys and SEA induced little or no IL-12(p70) (FIG. 7b). As indicated, the absolute amounts of cytokine secreted varied significantly from donor to donor, but the relative levels of the cytokines induced by the different stimuli was consistent. IL-10, a regulatory cytokine which is known to dampen both Th1 and Th2 responses in humans (Pulendran, B., J. L. Smith. G. Caspary, K. Brasel, D. Pettit, E. Maraskovsky, C. R. Maliszewski. 1999. Proc Natl Acad Sci USA 96:1036) was induced by Ec.LPS, flagellin and Pam3cys, and at lower levels by SEA (FIG. 7b). The pro-inflammatory cytokine, TNF-α was strongly induced by Ec.LPS and flagelhn, but induced at weaker levels by Pam3cys and SEA. Taken together, these data suggest that the different stimuli induce very distinct cytokine profiles from DCs. In particular, Pam3cys and SEA induce little or no IL-12(p70), relative to the TLR4 and TLR5 ligands. This impaired IL-12 induction was not a dose-related phenomenon, because even very high doses of Pam-3-cys and SEA, which induced high levels of CD83 on DCs, did not induce IL-12(p70).

Ec.LPS and Flagellin Induce Th1 Responses, but Pam3cys and SEA Bias the Response Towards the Th2 Pathway

Given these differences in cytokine secretion, whether DCs stimulated with the various stimuli were tested for the ability to induce different types of Th responses. DCs cultured for 48 h with the various stimuli were washed and cultured, at graded doses, with naive, allogeneic, CD4+ CD45RA+ CD45RO− T cells. After 5 days, the cultures were pulsed with tritiated thymidine (³[H]) for 12 h to measure the proliferation of T cells. As seen in FIG. 8a, in all cases, DCs induced efficient proliferation of T cells. The Th cytokines secreted in culture were determined by cytokine ELISA (FIG. 8b). DCs cultured in the absence of any stimuli induced less than 1000 pg/ml of the Th1 cytokine IFNγ, and 300-400 pg/ml of the Th2 cytokines IL-5 and IL-13, this profile being consistent with a Th0 response. However, DCs stimulated with Ec.LPS or flagellin induced approximately 4000 pg/ml of IFNγ and much lower levels of IL-5 and IL-13, a typical Th1 profile, this being consistent with the high levels of IL-12(p70) induced by these stimuli (FIG. 7A). In contrast, DCs stimulated with Pam3cys or SEA biased the response towards the Th2 pathway. In particular SEA induced a Th2 response, with less than 300 pg/ml of IFNγ (less than uncommitted DCs), but 800 pg/ml of IL-5, and 800 pg/ml of IL-13. Pam3cys induced approximately 2000 pg/ml of IFN-g, and high levels of IL-5 (600 pg/ml) and IL-13 (600 pg/ml). Interestingly, IL-4 could not be detected in any of the cultures, even with SEA, a classic Th2 stimulus, and even after restimulation of the T cells with anti-CD3+anti-CD28, or PMA+ionomycin. This is consistent with numerous other studies with human DCs (Kalinski, P., C. M. Hilkens, E. A. Wierenga, M. L. Kapsenberg. 2000. Immunol Today. 12: 561; Pulendran, B., K. Palucka, and J. Banchereau. 2001. Science. 293: 253; Shortman, K. and Y. J. Liu. 2002. Nature Reviews Immunol 2:151; Rissoan, M. C., V. Soumelis, N. Kadowaki, G. Grouard, F. Briere, R. de Waal Malefyt, Y. J. Liu. 1999 Science 283:1183) in which “classical Th2 responses,” have always been difficult to obtain in vitro. Nevertheless, these data suggest that TLR4 and TLR5 ligands induce uncommitted DCs to adopt a Th-1 inducing mode, but Pam3cys and SEA induce DCs that skew the response towards the Th2 end of the spectrum. This is underscored by the ratios of IFNγ/IL-5 or IFNγ/IL-13, which reflect the Th1/Th2 balance (FIG. 8c). While Ec.LPS and flagellin favor Th1 responses, Pam3cys and SEA clearly tilt the balance towards Th2 responses (FIG. 8b). This was not a dose-related phenomenon, because even very high doses of Pam-3-cys and SEA, which induced high levels of CD83 on DCs, did not induce Th1 responses.

With respect to whether Ec.LPS and flagellin induce Th1 responses via an IL-12(p70) mechanism, in all cases, IFNγ secretion was diminished, when a neutralizing antibody against IL-12 was used. Taken together, the present data suggest that TLR4 and TLR5 ligands induce Th1 responses via IL-12(p70), but TLR2 ligands or SEA, induce Th2/ThO responses, possibly via a default mechanism which fails to induce IL-2(p70).

Pam3cys and SEA Induce Enhanced ERK Signaling

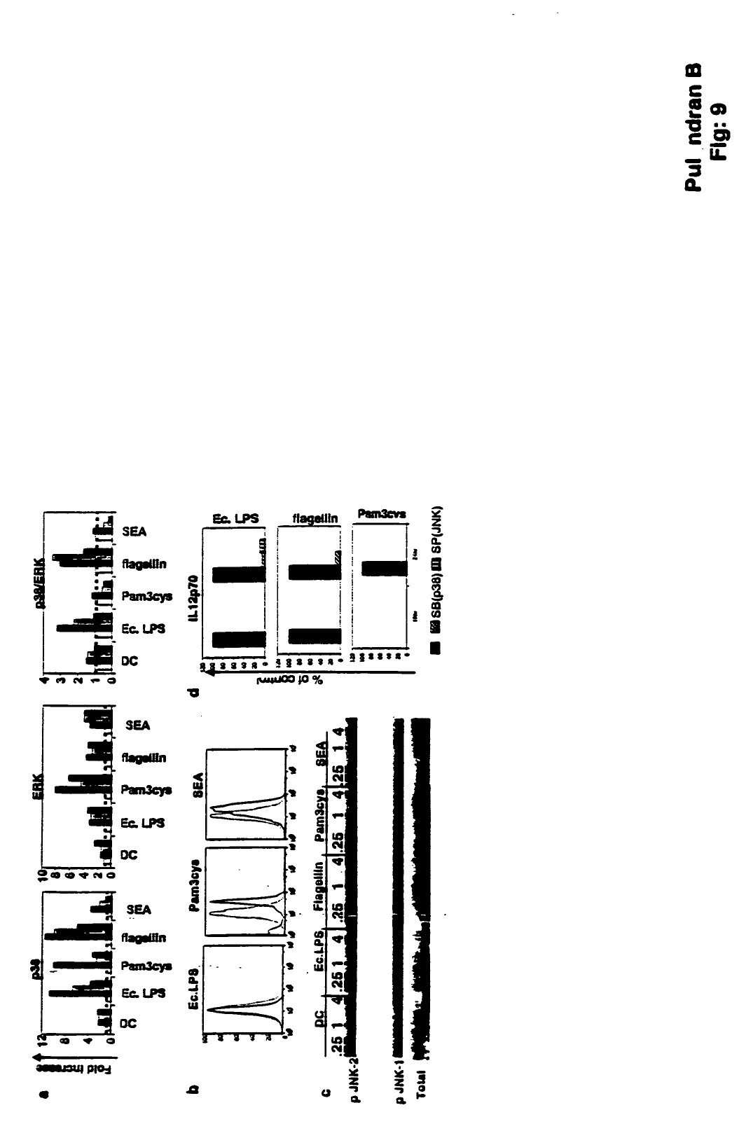

To investigate the potential intracellular signaling mechanisms which may mediate the different DC responses, the MAP-kinase signaling pathway, one of the most ancient signal transduction pathways in mammalian cells (Dong, C., R. J. Davis, R. A. Flavell. 2002. Annu. Rev. Immunol. 20: 55) was studied. MAP-kinases consist of three major groups—p38 MAP kinases, the extracellular signal-regulated protein kinases (ERK1 & 2), and the c-Jun NH₂-terminal kinases (JNK 1 & 2) (Dong, C., R. J. Davis, R. A. Flavell. 2002. Annu. Rev. Immunol. 20: 55). Previous reports indicate a critical role for MAP-kinases in regulating Th1/Th2 balance in T cells (Dong, C., R. J. Davis, R. A. Flavell. 2002. Annu. Rev. Immunol. 20: 55), and emerging evidence suggests a role for these proteins in regulating cytokine production from APCs (Yi, A. K., J. G. Yoon, S. J. Yeo, S. C. Hong, B. K. English, A. M. Krieg. 2002. J. Immunol. 168: 4711). The phosphorylation of p38, ERK1/2 and JNK1/2 in DCs stimulated with various stimuli was investigated. As shown in FIG. 9a, there were differences in the magnitude and duration of phosphorylation of the MAP kinases induced by the different stimuli. Ec.LPS, flagellin, as well as Pam-3-cys all induced enhanced phosphorylation of p38 MAP kinase, relative to unstimulated DCs, although there were some subtle differences in the duration of phosphorylation—Ec.LPS and flagellin induced enhanced duration of p38 phosphorylation, while Pam-3-cys did not (FIG. 9a). SEA was a very weak inducer of p38. In the case of ERK1/2 phosphorylation however, Pam3cys induced a much higher magnitude and duration of phosphorylation, (which was sustained even at 4 h) [FIG. 9a & b], compared to Ec.LPS and flagellin. SEA also induced ERK1/2 phosphorylation, which while weaker than that induced by Pam3cys, was sustained, at 4 h at levels significantly higher than background levels (up to 4-fold above baseline levels FIG. 9a & b). In contrast, LPS barely induces ERK phosphorylation above background levels, and gives at best a 2-fold increase. Importantly, the ratios of p38: ERK phosphorylation was much higher with Ec. LPS and flagellin stimulation, compared to the other groups [FIG. 9a].

The phosphorylation of JNK1 & 2 induced by the various stimuli was also examined. As shown in FIG. 9c, stimulation with Ec.LPS and flagellin induced higher levels of JNK 2, than stimulation with Pam3cys and SEA; however induction of JNK1 was more complex—while flagellin induced high levels, LPS, Pam-3-cys and SEA were very weak. These results demonstrate that distinct TLR ligands induce differences in the magnitude and duration of signaling of MAP-kinases in DCs.

Induction of IL-12(p70) is Enhanced by p38 and JNK1/2 Signaling, and Suppressed by ERK1/2 Signaling

The question of the roles played by p38, JNK1/2 and ERK1/2 in 1L-12(p70) induction by DCs was addressed using the well characterized, highly selective, synthetic inhibitors of p38 (SB203580, (Yi, A. K., J. G. Yoon, S. J. Yeo, S. C. Hong, B. K. English, A. M. Krieg. 2002. J. Immunol. 168: 4711)) ERK1/2 (UO126—a specific inhibitor of the upstream activators of MAP-kinase kinase 1 & 2 (MEK 1 & 2) (Yi, A. K., J. G. Yoon, S. J. Yeo, S. C. Hong, B. K. English, A. M. Krieg. 2002. J. Immunol. 168: 4711)], or JNK1/2 [SP600125 (Park, J. M., F. R. Greten, Z. W. Li, M. Karin. 2002. Science 297:2048)]. Blocking p38 or JNK1/2, largely abrogated IL-12(p70) production induced by Ec.LPS and flagellin (FIG. 9d). IL-12(p70) levels, after blocking with inhibitors, are expressed as a percentage of levels without inhibitor, (which is 100%). At 10 hrs, Pam-3-cys did not induce any IL-12, thus the value is 0%. At 24 hrs, Pam-3-cys induced 20-100 pg/ml of IL-12, and this is considered to be 100%.

Interestingly, blocking ERK1/2 activity significantly enhanced IL-12(p70) production induced by flagellin, Pam3cys, and Ec.LPS, suggesting an important role for ERK1/2 in the negative regulation of IL-12(p70) production [Table 1]. Despite the donor-to donor variation, as indicated in Table 1, there was a general trend for ERK inhibition to enhance IL-12, consistent with previous reports (Yi, A. K., J. G. Yoon, S. J. Yeo, S. C. Hong, B. K. English, A. M. Krieg. 2002. J. Immunol. 168: 4711). In the case of SEA, blocking ERK1/2 did not result in consistent increases in IL-12(p70), suggesting that additional mechanisms regulate the suppression of IL-12(p70), by SEA. Taken together, these data suggest that TLR4 and TLR5 agonists preferentially induce IL-12(p70) via a mechanism involving p38 and JNK1/2 phosphorylation. In contrast, Pam3cys and SEA induce enhanced duration or magnitude of ERK1/2 phosphorylation, a negative regulator of IL-12(p70).

TABLE 1
ERK is a negative regulator of IL-12(p70)

| Experiment | 1 | 2 | 3 | 4 |
|---|---|---|---|---|
| No inhibitor | 100 | 100 | 100 | 100 |
| Ec.LPS + U0126 | 110 | 158 | 178 | 131 |
| Pam3cys + U0126 | 447 | 215 | 100 | 118 |
| Flagellin + U0126 | 170 | 628 | 121 | 404 |

Pam3cys and SEA Induce Stabilization of Immediate Early Gene Product c-fos, which Regulates the Production of IL-12(p70)

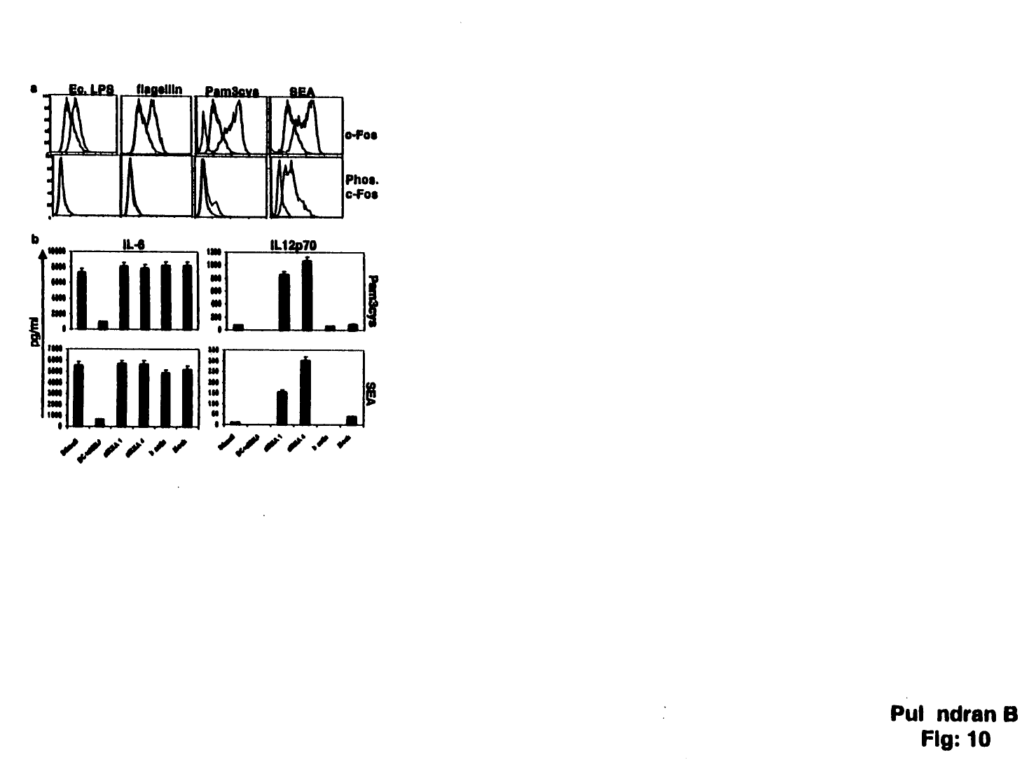

Whether enhanced ERK1/2 signaling results in suppression of IL-12(p70) was examined. Recent work suggests that sustained ERK signaling results in the phosphorylation and stabilization of the immediate early gene product c-Fos, in a fibroblast cell line (Murphy, L. O., S. Smith, R. H. Chen, D. C. Fingar, J. Blenis. 2002. Nat Cell Biol. 4:556). The kinetics and magnitude of expression of both total c-Fos and phosphorylated c-Fos (Phos. c-Fos) was determined, in DCs stimulated with the various stimuli, using antibodies directed against the two different forms of c-Fos. In FIG. 10a, the blue histograms represent expression levels in unstimulated, immature DCs, and the red histograms represent expression levels after stimulation with various stimuli (NO COLOR, BLACK AND WHITE FIGURES). As observed, all stimuli induced enhanced levels of c-Fos expression, relative to unstimulated DCs, and this c-Fos expression peaked after 2 hrs of stimulation. However, at this time point, the level of expression of total c-Fos, (as assessed by the mean-fluorescence intensity of staining), and fraction of cells expressing c-Fos, in DCs stimulated by Pam3cys or SEA is much greater, than in DCs stimulated with Ec.LPS, or flagellin. Consistent with this, the more stable, phosphorylated c-Fos, was not expressed in DCs stimulated with flagellin and Ec.LPS, but expressed at significant levels in DCs stimulated with Pam3cys and SEA [FIG. 10a]. Furthermore, c-Fos expression was maintained even at 4 hrs, in DCs stimulated with Pam3cys or SEA, but not with Ec.LPS, or flagellin. Therefore, stimulation of DCs by Pam3cys and SEA, which induce sustained duration of ERK1/2 signaling, results in the phosphorylation and stabilization of c-Fos.

The role of c-Fos in the regulation of IL-12(p70) was determined using the RNA interference (si RNA) technique (Dykxhoom, D. M., C. D. Novina, P. Sharp. 2003. Nat. Rev. Mol. Cell. Biol. 4: 457), to inhibit c-Fos expression in DCs. Five target sequences of 21 nucleotide si-RNA designed to target the c-fos gene, were selected from the Ambion website (http://www.ambion.com/techlib/misc/siRNA_finder.html). The transcription of siRNA and transfection in dendritic cells was done as per instructions from the Ambion kits. si RNA targeting c-fos gene, decreased the amount of corresponding protein, but did not lower DC viability. The induction of a “neutral” cytokine, such as IL-6 appeared to be unaffected by the reduction on c-Fos [FIG. 10b]. However, there was a profound enhancement of IL-12(p70) induction in response to Pam-3-cys, or SEA (FIG. 10b). There was a similar, although much less profound enhancement with LPS and flagellin. Strikingly, when c-fos activity is impaired, even a “classic Th2 stimulus, such as SEA, induces abundant IL-12(p70), and thus behaves as a Th1 stimulus. Taken together, these data suggest that c-Fos plays an important role in the negative regulation of IL-12(p70), and that stimuli such as Pam-3-cys and SEA, which appear to bias the Th response towards the Th2 pathway, induce enhanced levels of c-Fos expression in DCs.

In summary, these results suggest: (i) that activation of dendritic cells via TLRs, do not always result in Th1 responses (Medzhitov, R., and C. Janeway, Jr., 2000. Immunol Rev. 173:89; Sieling, P. A., W. Chung, B. T. Duong, P. J. Godowski, R. L. Modlin. 2003. J. Immunol. 170:194), but can also induce skew towards Th2 responses. (ii) a possible mechanism involving differential modulation of the threshold and duration of MAP-kinase signaling, which may mediate the distinct DCs responses triggered by the different TLR ligands. (iii) fundamental differences in the phosphorylation and stabilization of the early growth transcription factors c-Fos, which is phosphorylated and stabilized by enhanced ERK ½ signaling (Murphy, L. O., S. Smith, R. H. Chen, D. C. Fingar, J. Blenis. 2002. Nat Cell Biol. 4:556). Taken together, these data suggest an important role for c-Fos in regulating IL-12(p70) production within DCs. c-Fos belongs to the AP-1 family of transcription factors, (Murphy, L. O., S. Smith, R. H. Chen, D. C. Fingar, J. Blenis. 2002. Nat Cell Biol. 4:556).

Thus, E. coli LPS and flagellin, which trigger TLR4 and TLR5, respectively, instruct DCs to stimulate Th1 responses via IL-12(p70) production, which depends on the phosphorylation of p38 and JNK1/2. In contrast, the TLR2 agonist Pam3cys, and the Th2 stimulus, schistosome egg antigen (SEA): (i) barely induce IL-12(p70); (ii) stimulate sustained duration and magnitude of ERK1/2 phosphorylation, which results in stabilization of the transcription factor c-Fos, a suppressor of IL-12, and; (iii) yield a Th2 bias. Thus, distinct TLR agonists differentially modulate ERK signaling, c-Fos activity, and cytokine responses in DCs to stimulate different Th responses.

These data are consistent with an emerging paradigm suggests that signaling via distinct TLRs triggers qualitatively different responses from the innate immune system (Pulendran, B., K. Palucka, and J. Banchereau. 2001. Science. 293: 253; Pulendran B., et al. 2001 J. Immunol. 167: 5067; Re, F., and J. L. Strominger. 2001. J. Biol. Chem. 276:37692; Toshchakov, V., B. W. Jones, P. Y. Perera, K. Thomas, M. J. Cody, S. Zhang, B. R. Williams, J. Major, T. A. Hamilton, M. J. Fenton, S. N. Vogel. 2002. Nature Immuno.l 4: 392; Ito, T., R. Amakawa, T. Kaisho, H. Hemmi, K. Tajima, K. Uehira, Y. Ozaki, H. Tomizawa, S. Akira, S. Fukuhara. 2002. J. Exp. Med. 195:1507). This underscores novel therapeutic opportunities that will be gained by modulating TLRs, MAP-kinases, or early growth transcription factors, to manipulate adaptive immunity in the immune therapy of allergy, autoimmunity, transplantation and cancer.

### Example 3

Different Toll-Like Receptor Ligands Induce Dendritic Cell Activation and Immune Response In Vivo

The adaptive immune system can generate distinct classes of responses, but the mechanisms that determine this are poorly understood. This example demonstrates that different Toll-like receptor (TLR) ligands induce distinct dendritic cell activation and immune responses in vivo.

Material and Methods

Mice: C57BL/6 mice were purchased from The Jackson Laboratory (Bar Harbor, Me.). Male B6.PL-Thy 1^{a} (B6.PL) mice were purchased from Jackson or bred at the Rodent Vivarium of the Yerkes National Primate Center of Emory University (Atlanta, Ga.). B6129/F1/Tac (B6129) mice were purchased from Taconic, Germantown, N.Y. TLR-2 knockout mice (TLR2−/−) (Takeuchi, O. et al., Immunity, 11:443-451 (1999)), and MyD88 knockout (MyD88−/−) (Kawai, T., Adachi, O., Ogawa, T., Takeda, K. & Akira, S., Immunity, 11:115-122 (1999)) mice. OT-2 TCR transgenic mice (strain 426-6) (Barnden, M. J., Allison, J., Heath, W. R. & Carbone, F. R., Immunol. Cell. Biol., 76:34-40 (1998)), generated by Dr. W. Heath (Walter and Elisa Hall Institute, Melbourne, Australia) and Dr. F. Carbone (Monash University, Melbourne, Australia) were obtained from Dr. J. Kapp (Emory University, Atlanta, Ga.) and bred at the Yerkes Animal Facility. OT-1 TCR transgenic mice (Martin, S. & Bevan, M. J., Eur. J. Immunol., 27:2726-2736 (1997)) were obtained from Jackson Laboratories, and bred at the Yerkes Vivarium. All mice were aged 6-10 weeks. All animal studies were approved by the Institutional Animal Care and Use Committee (Emory University, Atlanta, Ga.). For adoptive transfer studies, age-matched B6.PL recipients were given 2.5×10⁶ of either OT-2 or OT-1 TCR transgenic T cells i.v.

Microbial Stimuli: Highly purified E. coli LPS (Strain 25922) and P. gingivalis LPS (A7436) were generous gifts from T. Van Dyke (Boston University School of Medicine, Boston, Mass.). Pam₃cys-Ser-Lys₄ (Pam₃cys) was obtained from G. Jung (Eberhard Karls Universitat Tüibingen, Tübingen, Germany) and reconstituted in endotoxin-free water. All antigens were sonicated before use.

Injections: B6.PL mice reconstituted with OT-2 TCR transgenic T cells were injected i.p with 50 μg MHC Class 1′-restricted OVA peptide (ISQVHAAHAEINEAGR; OVA₃₂₃₋₃₃₉) in PBS alone, or PBS containing either 25 μg E. coli LPS or 50 μg Pam₃cys. OT-1 TCR transgenic T cell-reconstituted B6.PL mice were injected in a similar fashion except with 50 μg MHC Class I-restricted OVA peptide (SIINFEKL; OVA₂₅₇₋₂₆₄). The OVA peptides were obtained from BioSynthesis, Inc (Lewisville, Tex.) and from Dr. Brian Evavold (Emory University, Atlanta, Ga.).

To investigate the effect of TLR ligands on DC in vivo, B6129 or TLR-2 knockout mice were injected with PBS containing either 25 μg E. coli LPS or 50 μg Pam₃cys. Six hours later, the spleens were removed and a small portion digested with Collagenase, Type 4 (1 mg/ml; Worthington Biochemical Corporation, New Jersey) in complete DMEM+2% FBS for 30 minutes at 37° C. The red blood cells were lysed and the cell suspension washed twice prior to analysis of cell surface expression of activation markers by flow cytometry.

Flow cytometry: All antibodies used were from BD PharMingen (San Diego, Calif.). For analysis of activation of DC after injection of TLR ligands in vivo, RBC-lysed, collagenase-digested spleen cells were incubated at 4° C. with FITC-conjugated CD11c, PE-conjugated CD11b and either biotin-labeled CD86 or MHC Class II followed by labeling with streptavidin APC. For analysis of OT-2 cells, cell suspensions prepared from spleen cells were incubated at 4° C. with APC-conjugated CD4 and PE-conjugated Thy 1.2. OT-1 cells were analyzed in a similar fashion, but with APC-conjugated CD8 and PE-conjugated Thy 1.2 antibodies.

Four days after in vivo priming with OVA peptide, OVA peptide and E. coli LPS or OVA peptide and Pam₃cys, RBC-depleted spleen cells were cultured in triplicate in 96 round-bottomed plates (1×10⁶ cells/well) in complete DMEM+10% FBS together with different concentrations of OVA peptide. Proliferative responses were assessed after 72 hours of culture in a humidified atmosphere of 5% CO₂ in air at 37° C. Cultures were pulsed with 1 μCi [³H]thymidine for 12 hours and incorporation of the radionucleotide was measured by β-scintillation spectroscopy. For cytokine assays, aliquots of culture supernatants were removed after 90 hours, pooled and assayed for the presence of IL-4, IL-5, IL-13 and IFN-γ by ELISA.

Measurement of cytokine production: IL-4, IL-5, and IFNγ were quantitated by ELISA sets from BD PharMingen, and IL-13 was measured by an ELISA kit from R&D Systems (Minneapolis, Minn.).

Results

E. coli LPS and Pam-3-cys Activate Splenic CD11c+CD11b- and CD11c+CD11b+DC Subsets In Vivo

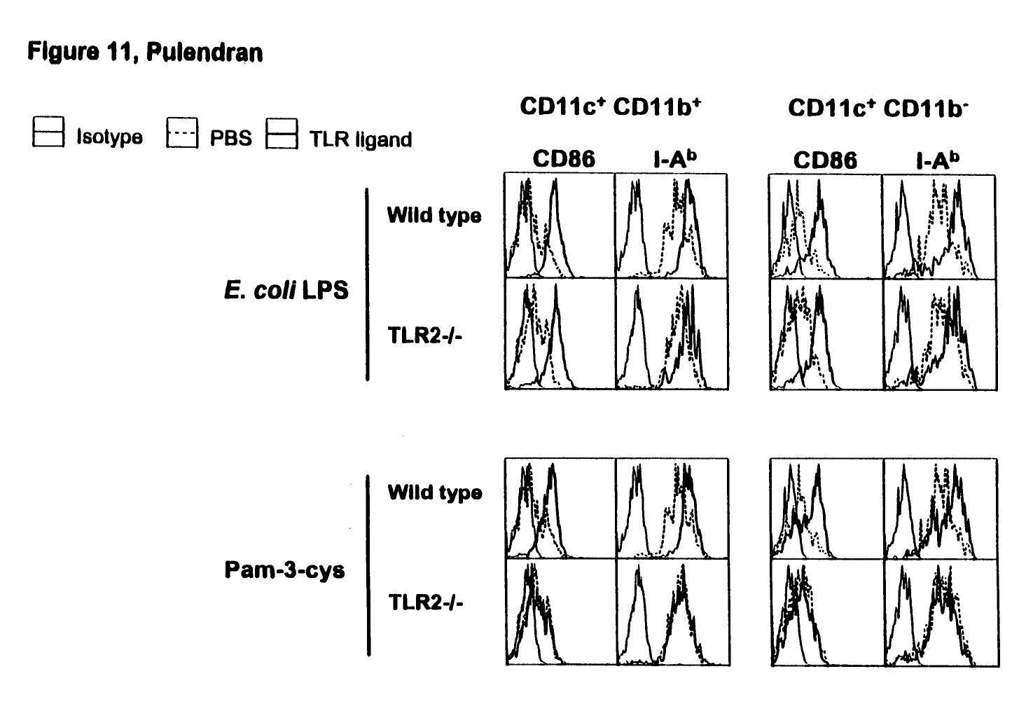

Whether TLR-4 and TLR-2 ligands could activate splenic DC subsets in vivo, was determined by injecting the ligands intravenously into wild type or TLR-2 deficient mice, and examining the microenvironmental localization of DCs, and their expression of costimulatory molecules, 4 or 6 h after injection. As shown in FIG. 11A, E. coli LPS and Pam-3-cys induce equivalent up-regulation of CD86 and MHC class II(1-A^{b}) on both CD11c+ CD11b+ and CD11c+ CD11b− DCs in wild type mice. In TLR-2 deficient mice, the induction of CD86 and I-A^{b} by Pam-3-cys was severely impaired, but the effects of E. coli LPS were unaffected. Therefore, the synthetic molecule Pam-3-cys appears to activate DCs in vivo, via TLR-2.

E. coli LPS and Pam-3-cys Induce Different Classes of Antigen-Specific CD4+ T Cell Responses In Vivo

Whether different TLR ligands stimulated different types of CD4+ T-helper immune responses in vivo was addressed, using OVA-specific, MHC class II-restricted (1-A^{b}), αβ TCR transgenic mice (OT-2 mice) (Barnden et al, 1998). In these mice, the CD4+ OVA-specific T cells express Vα2 and Vβ5, and recognize the amino acid 323-339 peptide fragment (hereafter denoted as OVA₃₂₃₋₃₃₉) from OVA. TCR transgenic T cells were adoptively transferred into Thy-1 congenic B6.PL.Thy-1^{a} (B6.PL) mice, such that they constituted a small, but detectable proportion of all T cells (Kearney, E. R., Pape, K. A., Loh, D. Y. & Jenkins, M. K., Immunity, 1:327-339 (1994); Pape, K. A. et al., Immunol. Rev., 156:67-78 (1997a); Pape, K. A., Khoruts, A., Mondino A. & Jenkins, M. K., J. Immunol., 159:591-598 (1997b)). In this system, the fate of OVA-specific, transgenic T cells was followed using the Thy-1.2 antibody, which stains the transferred cells, but not the host cells. Cells with the phenotype Thy-1.2+ CD4+ Vα2+ Vβ5+ are considered OVA-specific CD4+ T cells. In some of the experiments, Thy-1.2 was used in combination with CD4, to detect the OVA-specific T cells.

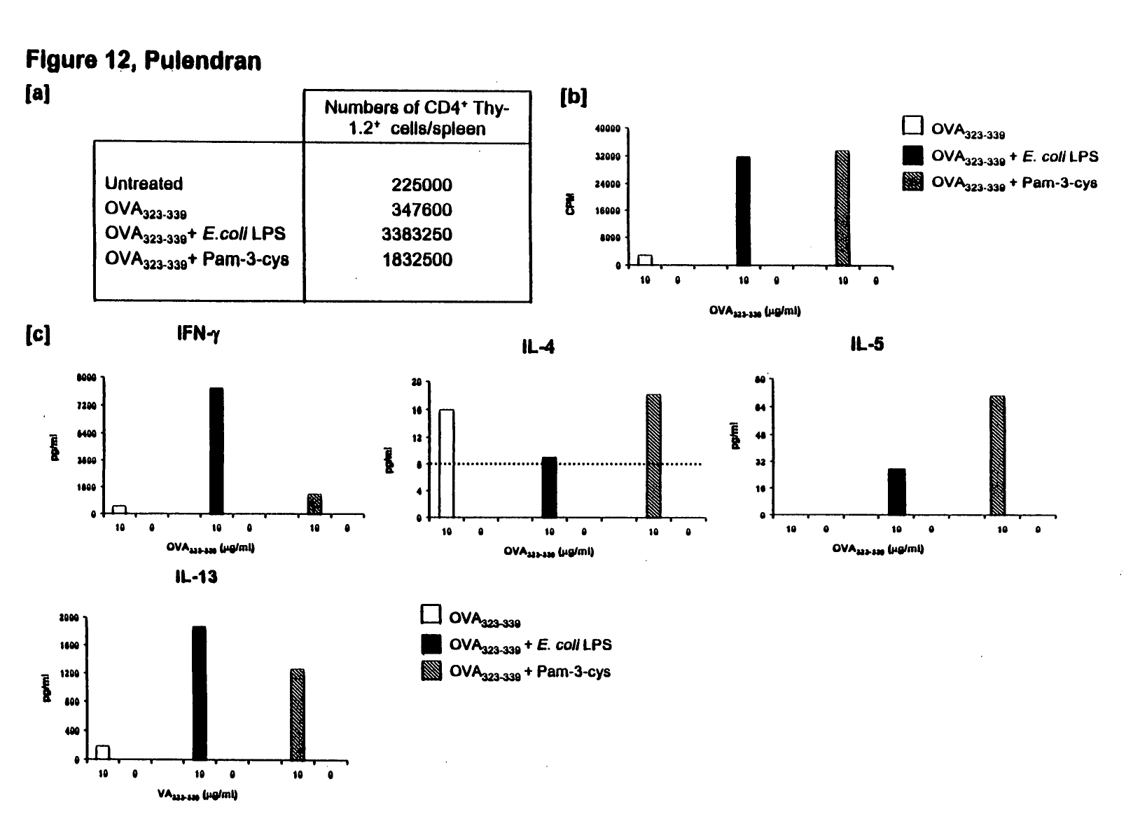

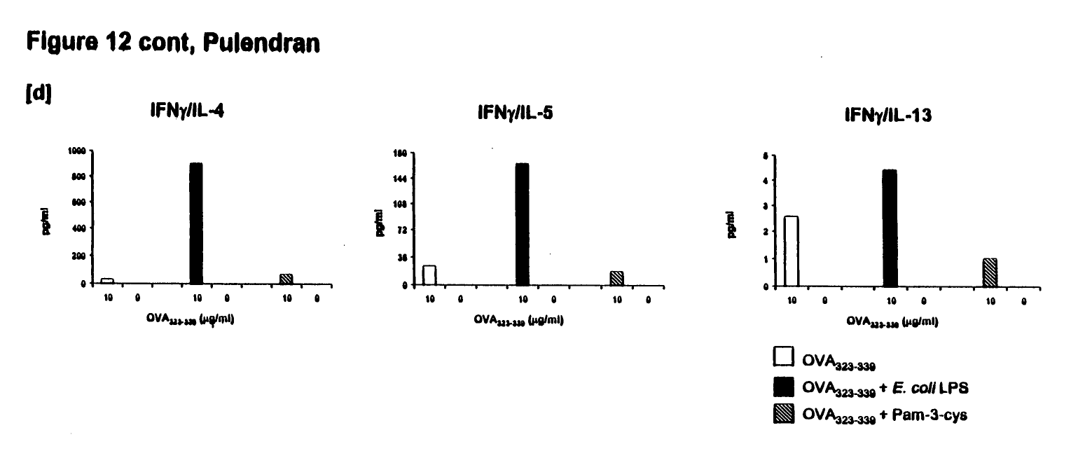

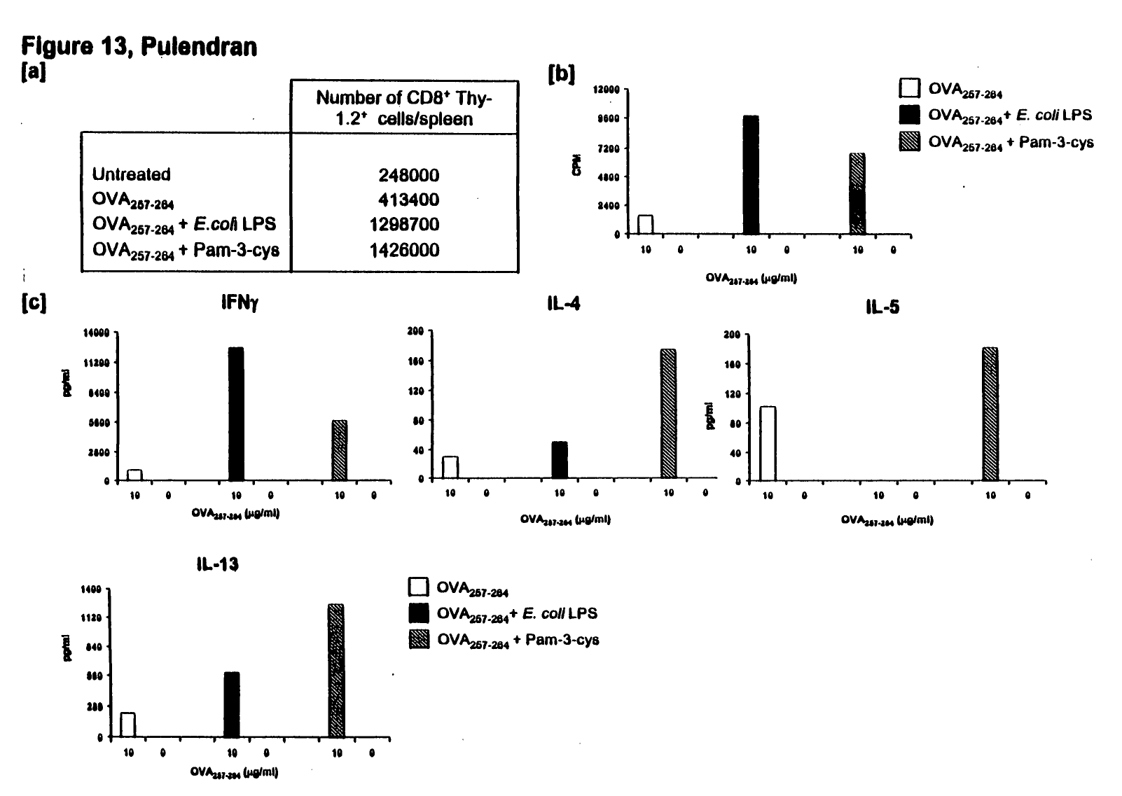

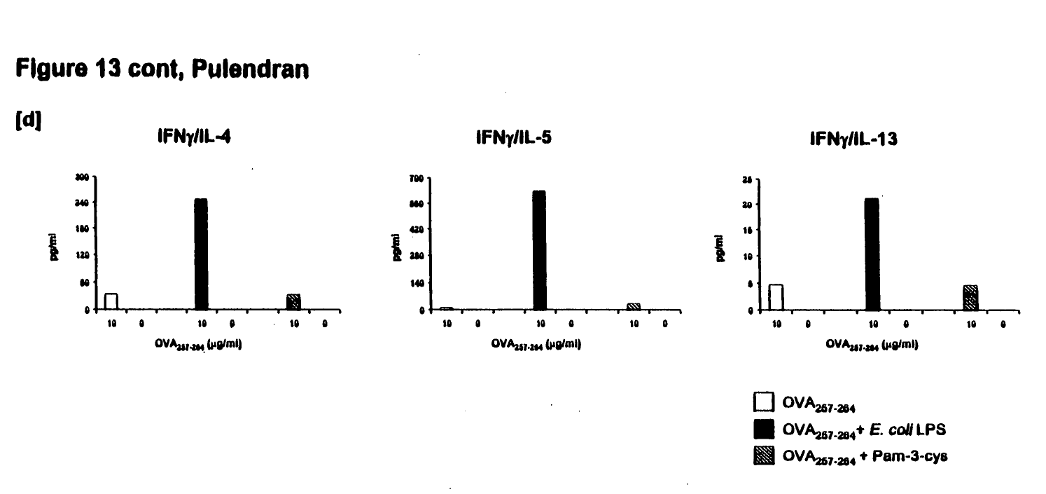

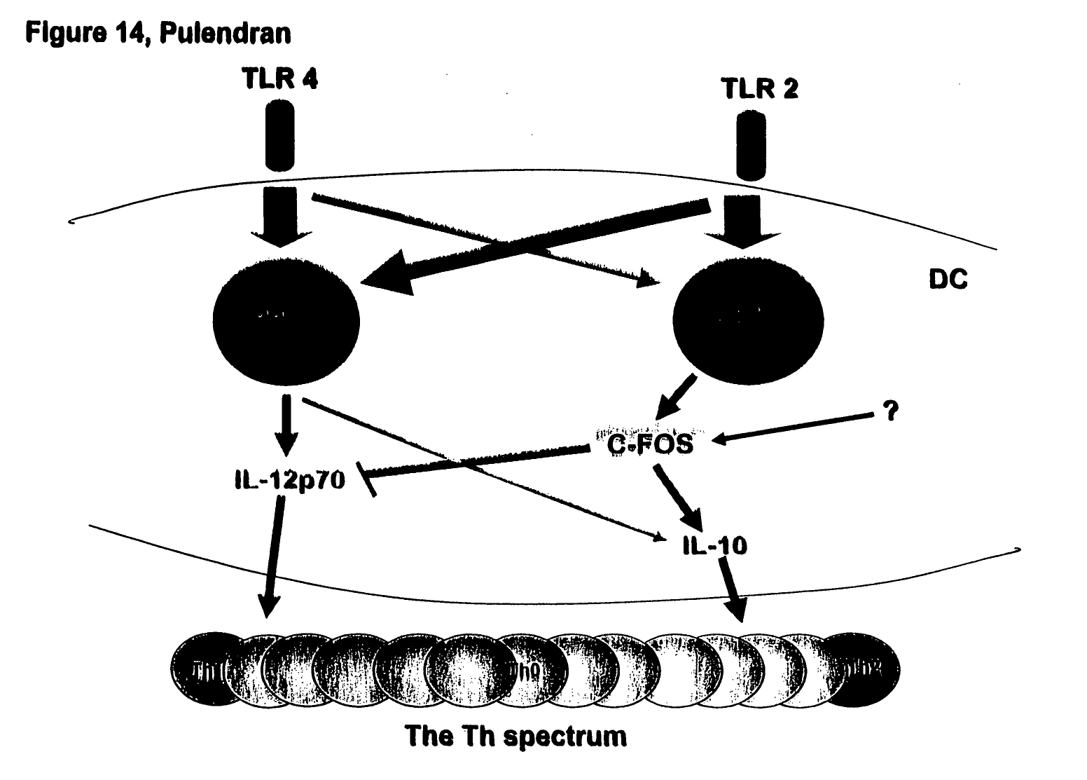

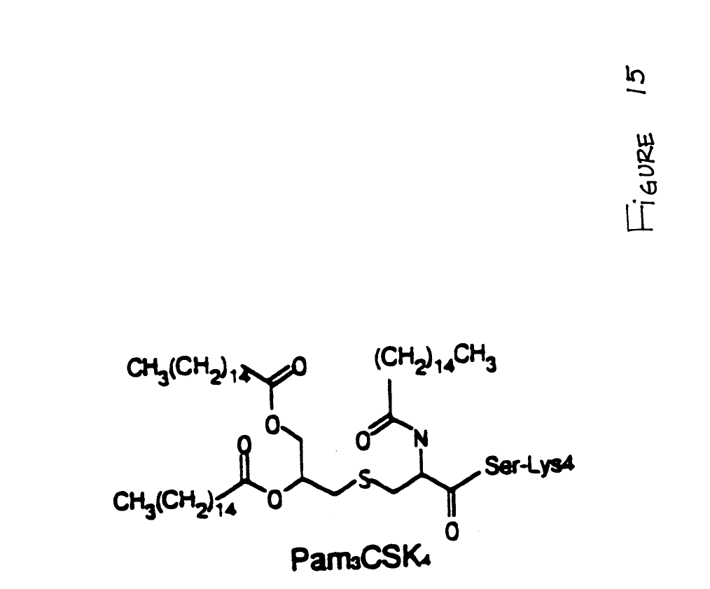

The reconstituted mice were injected with 50 μg of OVA₃₂₃₋₃₃₉ peptide alone, or OVA₃₂₃₋₃₃₉+E. coli LPS, or OVA₃₂₃₋₃₃₉+Pam-3-cys intraperitoneally (i.p). Injection of OVA₃₂₃₋₃₃₉ alone did not induce any significant clonal expansion of the CD4+ Thy-1.2+ cells in the spleens of mice (FIG. 12A). However, both E. coli LPS and Pam-3-cys significantly enhanced the clonal expansion of CD4+ Thy-1.2+ cells. Previous work has shown that productive T cell immunity is elicited only when the antigen is injected with an adjuvant, and that injections of soluble antigens only result in a transient and abortive clonal expansion, in which antigen-specific T cells cannot be efficiently restimulated in vitro, with protein or peptide (Kearney, E. R., Pape, K. A., Loh, D. Y. & Jenkins, M. K., Immunity, 1:327-339 (1994); Pape, K. A. et al., Immunol. Rev., 156:67-78 (1997a); Pape, K. A., Khoruts, A., Mondino A. & Jenkins, M. K., J. Immunol., 159:591-598 (1997b)). The in vitro proliferative capacity of OVA-specific T cells from the various cohorts of mice, was examined by culturing single cell suspensions of the spleen with varying concentrations of OVA. As shown in FIG. 12B, mice that received OVA₃₂₃₋₃₃₉+E. coli LPS or OVA₃₂₃₋₃₃₉+Pam-3-cys had greatly enhanced responses, compared to those that received OVA₃₂₃₋₃₃₉ peptide alone.

Cytokine production by antigen-specific T cells was measured by assaying the culture supernatants from the cultures described above for IFNγ, IL-4, IL-5, and IL-13. There were significant differences between mice injected with OVA₃₂₃₋₃₃₉ peptide alone, or OVA₃₂₃₋₃₃₉+E. coli LPS, or OVA₃₂₃₋₃₃₉+Pam-3-cys [FIG. 12C]. In cultures from mice injected with OVA₂₅₇₋₂₆₄ peptide alone, there was little, if any, IFNγ, IL-4, IL-5, or IL-13. In contrast, and consistent with previous reports (Pulendran, B. et al., J. Immunol., 167:5067-5076 (2001b); Pape, K. A., Khoruts, A., Mondino A. & Jenkins, M. K., J. Immunol., 159:591-598 (1997b)), in cultures from mice injected with OVA₃₂₃₋₃₃₉+E. coli LPS, there were high levels of IFNγ, and low levels of IL-4 (˜8 pg/ml) and IL-5 (˜30 pg/ml). In addition, there was a significant level of IL-13. Considering that the sensitivity of the cytokine ELISA assay is 8 pg/ml [dotted line, FIG. 12c], the levels of 1L-4 and IL-5 induced by E. coli LPS are either below or barely above the threshold of detection, and thus E. coli LPS biases the response towards the Th1 pathway. This Th1 induction by E. coli LPS was dependent on IL-12(p70), since its neutralization, in vivo, with an antibody, largely impaired IFNγ production. However, the induction of significant levels of IL-13, as observed previously (Pulendran, B. et al., J. Immunol., 167:5067-5076 (2001b)), suggests that the response induced does not fit the “canonical Th1 profile.” In striking contrast to this response, in cultures from mice injected with OVA₃₂₃₋₃₃₉+Pam-3-cys, there was much lower levels of IFNγ, significantly higher levels of IL-5 (˜70 pg/ml) and 1L-4 (˜30 pg/ml), and similar levels of IL-13 as that induced by E. coli LPS. Although the absolute levels of cytokines induced varied from experiment to experiment, in every experiment Pam-3-cys induced a much greater Th2 bias than LPS. Thus, Pam-3-cys induces a response in which there is a preferential bias towards the Th2 pathway, consistent with its effective induction in DCs of IL-10, a Th2-inducing cytokine (Manickasingham, S. P., Edwards, A. D., Schulz, O. & Reis e Sousa, C., 2003, Eur. J. Immunol., 33:101-107 (2003)). This response is unlikely to represent a T-regulatory response, since Parn-3-cys was able to induce significant clonal expansion and in vitro proliferation [FIGS. 12 & 13]. Thus, although neither stimulus induces canonical Th1 or Th2 responses, they exert strikingly different influences on modulating the Th1/Th2 balance. This is further illustrated by the ratios of Th1: Th2 cytokines induced by the E. coli LPS versus Pam-3-cys [IFNγ: IL-4, 975 vs 40; IFNγ: IL-5, 162 vs 18; IFNγ: IL-13, 4.5 vs 1.2] (FIG. 12D).

E. coli LPS and Pam-3-cys Induce Distinct Types of Antigen-Specific CD8+ T Cell Responses In Vivo

The propensities of E. coli LPS and Pam-3-cys to stimulate different classes of Th responses in vivo, was investigated, using OT-1 mice (H-2 K^{b}-restricted, OVA-specific TCR transgenic mice) (Martin, S. & Bevan, M. J., Eur. J. Immunol., 27:2726-2736 (1997)) to determine whether these stimuli could induce distinct types of CD8+ T cell responses in vivo. A total of 2.5×10⁶ spleen cells from OT-1 mice were transferred into B6.PL (Thy1.2) mice. Cohorts of host mice were injected with either OVA₂₅₇₋₂₆₄+E. coli LPS or OVA₂₅₇₋₂₆₄+Pam-3-cys. Clonal expansion of OVA-specific CD8+ T cells (CD8+ Thy-1.2+) was assessed by flow cytometry (FIG. 13A). Both E. coli LPS+OVA₃₂₃₋₃₃₉ and Pam-cys+OVA₂₅₇₋₂₆₄ enhanced the clonal expansion of OVA-specific CD8+ T cells.

The in vitro proliferative capacity of the OVA-specific CD8+ T cells from the various cohorts of mice, was examined by culturing single cell suspensions of the spleen with varying concentrations of OVA₂₅₇₋₂₆₄. As shown in FIG. 13B, mice that received an injection of either E. coli LPS+OVA₂₅₇₋₂₆₄, or Pam-3-cys+OVA₂₅₇₋₂₆₄ had greatly enhanced responses, compared with those that received OVA₂₅₇₋₂₆₄ alone.

Cytokine production by antigen-specific T cells was measured by assaying the culture supernatants from the cultures described above for IFNγ, IL-4, IL-5 and IL-13 (FIG. 13C). There were significant differences between mice injected with OVA₂₅₇₋₂₆₄ peptide alone, or OVA₂₅₇₋₂₆₄+E. coli LPS, or OVA₂₅₇₋₂₆₄+Pam-3-cys. In cultures from mice injected with OVA₃₂₃₋₃₃₉ peptide alone, there was little, if any, IFNγ, IL-4, IL-5 or IL-13. In contrast, and consistent with previous reports (Pulendran, B. et al. J. Immunol., 167:5067-5076 (2001); Pape, K. A., Khoruts, A., Mondino A. & Jenkins, M. K., J. Immunol., 159:591-598 (1997)), in cultures from mice injected with OVA₂₅₇₋₂₆₄+E. coli LPS, there were high levels of IFNγ, and much lower levels of IL-4, IL-5 and IL-13. Thus, E. coli LPS appears to skew the Tc balance towards the Tc1 pathway. However, compared with the cultures from the mice injected with E. coli LPS, in cultures from mice injected with OVA₂₅₇₋₂₆₄+Pam-3-cys, there were lower levels of IFNγ, but higher levels of IL-4, IL-5 and IL-13. Therefore, Pam-3-cys appears to shift the balance towards the Tc2 pathway. This is further illustrated by the ratios of Tc1: Tc2 cytokines induced by the E. coli LPS versus Pam-3-cys [IFNγ: IL-4, 260 vs 28; IFNγ: IL-5, 685 vs 21 IFNγ: IL-13, 22 vs 4] (FIG. 13D).

Discussion

Thus, E. coli LPS (TLR-4 stimulus), activates DCs to Th1 and Tcl responses. In contrast, Pam-3-cys (TLR-2 stimulus) favors Th2 and Tc2 responses. Therefore, different TLR ligands induce distinct cytokines and signaling in DCs, and differentially bias T-helper responses in vivo.

The present data suggest that distinct TLR ligands can elicit diverse signaling pathways and cytokine profiles, which regulate the Th1/Th2 balance. There is now emerging evidence that signaling via different TLRs can yield distinct functional responses. For example, a recent study suggests that activating murine macrophages in vitro via TLR 4 and TLR 2 yields IL-1α and TNF-α, respectively (Jones, B. W. et al., J Leukoc. Biol., 6:1036-1044 (2001)), although the functional significance of this difference in IL-1 and TNF production is not clear. Second, a study by Re and Strominger suggests that activating human monocyte-derived DCs with different TLR-agonists induces distinct cytokines, but the consequences of these different cytokines on adaptive immunity, or the signaling mechanisms which elicit the production of the different cytokines are not known (Re, F. & Strominger J. L., J. Biol. Chem., 276:37692-37699 (2001)). Third, Hirschfeld et al. demonstrated that highly purified P. gingivalis LPS signals through TLR 2 and induces a distinct profile of cytokines from murine macrophages in vitro, compared with E. coli LPS (Hirschfeld, M. et al., Infect. Immun., 69:1477-1482 (2001)). Consistent with this observation, these data suggests that highly purified P. gingivalis LPS fails to induce IL-12(p70) in murine DCs, and induces Th2 responses (Pulendran, B. et al., J. Immunol., 167:5067-5076 (2001b)). Fourth, it has recently been shown that triggering TLR 4, but not TLR 2 results in STAT-1 phosphorylation and IFN-α production (Toshchakov, V. et al., Nat. Immunol., 4:392-398 (2002)). Finally, a recent report suggests that triggering human monocyte-derived DCs or plasmacytoids DCs through TLR 7 results in differential induction of IL-12 and IFN-α (Ito, T. et al., J. Exp. Med., 195:1507-1512 (2002)).

Individual T cells display a rather complex spectrum of cytokine profiles, in which the canonical Th1 and Th2 cells represent only the very extreme ends of an axis (Kelso A., Immunol. Today, 16:374-379 (1995)). The data in this Example suggest that neither stimulus induces a typical Th1 or Th2 response. Rather, each stimulus appears to modulate the response towards opposite ends of the Th1/Th2 spectrum (FIG. 14).

The present data offer some novel perspectives on the mechanisms which underlie the complex decision-making processes which determine the striking diversity of immune responses generated against different microbes. Furthermore, the data underscore novel therapeutic opportunities that will be gained by modulating critical parameters (e.g. TLRs, MAP-kinases, transcription factors) in the immune therapy of cancer, allergy, autoimmunity and transplantation.

A number of embodiments of the invention have been described. Nevertheless, it will be understood that various modifications may be made without departing from the spirit and scope of the invention. Accordingly, other embodiments are within the scope of the following claims.

1-121. (CANCELLED)

122. A method for regulating a Th2 immune response, comprising contacting a TLR-2 expressing cell with an amount of an agent effective to induce signaling of an ERK 12 pathway in the cell so as to regulate the Th2 immune response, wherein the agent is an (a) agonist of a TLR receptor, an ERK ½ pathway, or a c-fos pathway or (b) an antagonist of a TLR receptor, an ERK ½ pathway, or a c-fos pathway thereby regulating the Th2 immune response.

123. The method of claim 122, wherein the induced signaling of the ERK ½ pathway produces phosphorylated ERK ½.

124. A method for regulating a Th2 immune response, comprising contacting a TLR-2 expressing cell with an amount of an agent effective to increase expression of c-fos so as to regulate the Th2 immune response, wherein the agent is an (a) agonist of a TLR receptor, an ERK ½ pathway, or a c-fos pathway or (b) an antagonist of a TLR receptor, an ERK ½ pathway, or a c-fos pathway thereby regulating the Th2 immune response.

125. The method of claim 124, wherein the increase in the expression of c-fos is an increase in the level of c-fos RNA, an increase in the level of c-fos protein, an increase in the level of c-fos phosphorylation, an increase the level of c-fos protein stability, or an increase in the level of c-fos post-translational modification.

126. The method of claim 122 or 124, wherein the Th2 immune response is enhanced and the number of functional Th2 cells is increased.

127. The method of claim 122 or 124, wherein the TLR-2 expressing cell expresses TLR-2 and TLR-1, or TLR-2 and TLR-6.

128. The method of claim 122 or 124, wherein the TLR-2 expressing cell is a dendritic cell, an immature dendritic cell, a mature dendritic cell, a monocyte derived dendritic cell, a plasmacytoid dendritic cell, a mast cell, or a bone marrow precursor cell.

129. The method of claim 122 or 124, wherein the TLR-2 expressing cell is a bovine, porcine, murine, equine, canine, feline, simian, human, ovine, piscine or avian cell.

130. The method of claim 122 or 124 wherein the agonist of the TLR receptor is a peptidoglycan, zymosan, bacterial lipopeptide, lipoteichoic acid, lipoarabinomannan, phenol-soluble modulin, glycoinositolphospholipids, glycolipids, porins, LPS from Leptospira interrogens, LPS from Porphyromnas gingivalis, HSP70, non-toxic cholera toxin, and Candida albicans toxin.

131. The method of claim 122 or 124, wherein the agonist of a TLR receptor, the agonist of the ERK ½ pathway, or the agonist of the c-fos pathway is naturally-occurring or synthetic.

132. The method of claim 130, wherein the bacterial lipopeptide is a diacylated or triacylated lipopeptide.

133. The method of claim 132, wherein the diacylated lipopeptide is Macrophage Activating Lipopeptide 2 kilo-Dalton from Mycoplasma fermantans, MALP2, Pam2CSK4, Pam2CGNNDESNISFKEF, or Pam2CGNNDESNISFKEK-SK4.

134. The method of claim 132, wherein the triacylated lipopeptide is Pam3cys-Ser-(Lys)₄.

135. The method of claim 122 or 124, wherein the agonist of the ERK ½ pathway is CpG DNA.
